(19) United States
(12) Patent Application Publication
(10) Pub. No.: US 2025/0333436 A1
(43) Pub. Date: Oct. 30, 2025

(54) Aqueous Solid Phase Peptide Synthesis

(71) Applicant: PolyPeptide Laboratories Holding (PPL) AB, Limhamn (SE)

(72) Inventors: Jan Pawlas, Frederiksberg C (DK); Olivier Ludemann-Hombourger, Rohrwiller (FR)

(21) Appl. No.: 18/579,577

(22) PCT Filed: Jul. 14, 2022
(86) PCT No.: PCT/SE2022/050713

§ 371 Date: Jan. 16, 2024

(30) Foreign Application Priority Data
Jul. 16, 2021 (SE) 2130202-1

(51) Int. Cl. C07K 1/04; C07K 1/06; C07K 1/08; C07K 1/10

(52) U.S. Cl. CPC C07K 1/042; C07K 1/063; C07K 1/08; C07K 1/10

(57) ABSTRACT

The present invention relates to a solid phase peptide synthesis (SPPS) where the coupling of amino acids is performed in an aqueous solution comprising at least one organic co-solvent miscible with water. The aqueous solution is capable of sufficiently solubilize the activated Fmoc-α-amine protected amino acid or activated Fmoc-α-amine protected peptide fragment, and where the resin is capable of swelling in the presence of the aqueous solution above about 4 mLg−1. The invention also encompasses a method for regeneration of spent aqueous solution from the SPPS.

### FIELD OF THE INVENTION

The present invention relates to a solid-phase peptide synthesis (SPPS) method comprising the use of an aqueous solution during peptide coupling and removal of temporary α-amine protecting groups. The aqueous solution comprising a co-solvent is capable of solubilizing appropriately activated Fmoc-protected amino acids and peptide fragments. Furthermore, the base resin has the characteristic to swell at least 4 mLg⁻¹ in the aqueous solution. The aqueous solution is also suitable for re-generation, thus, the invention also encompasses a method for regenerating spent aqueous solutions and spent solution for use in SPPS.

### BACKGROUND

Peptides are organic molecules comprising naturally occurring and modified amino acids comprising from a few amino acids up to around 60 amino acids. Proteins, as peptides, also comprise amino acids. One metric used to delineate proteins from peptides is the number of amino acids. Although there is no consensus as to the boundary, a molecule with over 60 amino acids usually is referred to as a protein, whereas molecules up to about 60 amino acids are denoted as peptides. The method and regeneration disclosed herein relate to peptides comprising up to about 60 amino acids.

Amino acids of a peptide are linked via amide bonds often referred to as peptide bonds. The amide bonds are typically formed by a condensation reaction of a carboxyl group of one amino acid with the amino groups of another amino acid, yet other chemical reaction mechanisms can also form amide bonds. Useful methods for the synthesis of peptides include liquid phase peptide synthesis (LPPS) and solid phase peptide synthesis (SPPS) and combinations of LPPS and SPPS. The SPPS method was pioneered by Bruce Merrifield in the 1960′. SPPS allows for a convenient assembly of a peptide chain by the successive reaction of amino acids derivatives on an insoluble support.

The coupling of a peptide residue to an insoluble support allows the introduction of several important physical and chemical process operations, such as filtering and washing steps, between the reaction step for formation of amide bonds.

The formation of amide-bonds between amines and carboxylic acids is not thermodynamically favored. Without the presence of compounds influencing the reactivity of the α-carboxyl group of the amino acid to be coupled (such as coupling agents, CAs), the amide-bond formation is often too slow for commercial applications. A further important methodology for driving the reaction (peptide-bond formation) has been and still is to push the reaction to the product side by applying excess reactants and reagents. The use of excess reactants and reagents is rendered feasible because the growing peptide is bound to an insoluble support/resin, enabling excess of reactants and reagents to be easily removed by e.g. filtering.

While SPPS allows for the application of excess reactants creating thermodynamically favorable conditions for increasing yield, this strategy is simultaneously also responsible for excessive consumption of reactants. The consecutive extension of the peptide bound to the support by a series of cycles, each cycle containing and at least one washing unit operation, and the removal of the reaction solution creates a significant volume of spent reaction solution.

In SPPS the α-amine of amino acids must be protected before the formation of the amide-coupling to the peptide fragment bound to the support otherwise it would be impossible to control the formation of the target peptide due to uncontrolled self-polymerization. Furthermore, reactive side chains of amino acids, notably side chains comprising amine-groups, are customarily also protected.

The dominant strategy since several years in SPPS has been to block the α-amine of amino acids with the 9-fluorenylmethoxycarbonyl (Fmoc) group (J. Pept. Sci. 2003 Sep 9(9): 545-52). The Fmoc group requires only mild/moderate bases for removal. Other reactive groups of amino acids, such as functionalized side chains, are typically protected by acid labile protective groups such as trityl (Trt) and tert-butyl (tBu). The Fmoc strategy allows that side chain protective groups are cleaved simultaneously when the crude target peptide is cleaved off from the resin using acid conditions, usually strong acidolysis preferably with trifluoroacetic acid (TFA).

The covalent linkage of Fmoc to the α-amine of amino acids has an impact on the solubility of the Fmoc protected amino acid. Fmoc comprises the hydrophobic aromatic fluorene moiety. Thus, the Fmoc protective amino acid is rendered more hydrophobic than the un-protected amino acid. The reaction solution should be able to solubilize activated Fmoc protected amino acids.

A further important aspect of SPPS is the appropriate swelling of the peptide resin. The solvent has a significant impact on the solvation of the resin. Thus, care must be exercised in the selection of the solvent and resin in respect of swelling. Additionally, the solvent (reaction solution) must also satisfy several other criteria/dimensions such as solubilization of the protected amino acids, either as such or in their activated forms. For SPPS to be successfully implemented the reaction solution and the resin (to mention just a few) need to fulfill multiple criteria in relation to several factors of which several have been articulated herein. The challenge is that an improvement in one dimension (e.g. solubilization) may exhibit the deterioration of other significant dimensions (e.g. resin swelling properties). To find the proper combination of reaction solution, α-amide protection group and resin is not straightforward (J. Pept. Sci. 2016; 22, 4-27).

As the solubilization of Fmoc-protected amino acids is important for reasons explained, solvents in Fmoc SPPS have, to a certain extent, been selected based on their ability to properly solubilize Fmoc protected amino acids. As presented, the Fmoc group is hydrophobic and rendering the Fmoc protected amino acid increasingly hydrophobic. The solvents of choice are selected from organic polar aprotic solvent, predominantly methylene chloride (DCM) N-methylpyrrolidone (NMP), NN-dimethylformamide (DMF) and NN-dimethylacetamide (DMA). All the commonly applied organic polar aprotic solvent in SPPS are to an extent carcinogenic, mutagenic or interfere in the reproduction (CMR substances).

For reasons presented it would be desirable to reduce the volume of organic solvents for SPPS by the partial replacement with water. It would also be desirable that any organic co-solvents used are not hazardous to the human health, for example belonging to the aforementioned CMR substances such as DMF, NMP, DCM or DMA. Furthermore, it would be desirable to replace part of the organic solvent with water while applying Fmoc-amino acid strategy.

US 2017/0218010 A1 discloses a SPPS process using a solvent of water or alcohol or a mixture of water or alcohol. Fmoc and Boc amino acid protection groups are hydrophobic and not soluble in water. The introduction of Fmoc and Boc as α-amino protection groups renders the amino acids more hydrophobic which is even compounded if reactive side groups are protected with groups with hydrophobic character. The proposition of US 2017/0218010 A1 is the modification of the α-amine protection group by introducing hydrophilic moieties rendering the protection group less hydrophobic. US 2017/0218010 A1 does not venture on the path elaborating on the solvent composition nor resin.

In a similar vein, Hojo et al (2003) explores the provision of a new water-soluble protection agent, 2-[phenyl(methyl)sulfonio]ethyl-4-nitro-phenylcarbonate tetrafluoroborate (Pms-ONp), for solid-phase peptide synthesis in aqueous solution. Amine protected amino acids are used in SPPS comprising a water-swellable crosslinked ethoxylate resin (CLEAR®) in the synthesis of Met-enkephalin. Hojo et al does not suggest an aqueous solution comprising an organic co-solvent miscible in water. The focus is on the provision of water-soluble protected amino acids which can be used with water as the solvent, by protecting the amino acids with water soluble 2-[phenyl(methyl)sulfonio]ethyl-4-nitro-phenylcarbonate tetrafluoroborate.

Furthermore, Hojo et al (2007) discloses an aqueous SPPS where organic solvents have been omitted using Fmoc protected amino acids. Fmoc is hydrophobic and renders an amino acid protected with Fmoc poorly soluble in an aqueous solution. The Fmoc protected amino acids are made more accessible for reacting with the resin-bound peptide fragment by transforming the Fmoc protected amino acids into a dispersion comprising polyethylene glycol (PEG). The dispersion of Fmoc protected amino acids are formed by subjecting an aqueous solution of PEG and Fmoc protected amino acid vigorous mixing using a planetary ball mill containing zirconium oxide beads. After extensive milling (495 rpm, 2 hours) the beads are removed and a dispersion is provided with a particle size of 265+/−10 nm. Instead of providing Fmoc protected amino acids in the form of dispersions, the present invention proposes a SPPS method where the coupling of amino acids is performed in an aqueous solution comprising at least one co-solvent and a resin capable of swelling more than 4 mL/g⁻¹ where the aqueous solution is capable of solubilizing Fmoc protected amino acids.

US 2012/0157563 A1 also explores amino acid protection groups which include a β unsaturated sulfone, such as Bsmoc (e.g. 1,1-dioxobenzo[b]thiphene-2 ylmethyloxycarbonyl) and Nsmoc (e.g. 1,1-dioxonaptho[1,2-b]thiophene-2-methyloxycarbonyl) and the deprotection of amino acids comprising said protection groups and subsequent washing of the deprotected peptides bound to a solid support with a solution of water, ethanol or an aqueous solution of ethanol. The pivotal aspect is the provision of water-soluble protection groups.

JP 2008056577 A discloses a solid phase peptide synthesis protocol comprising the use an aqueous solvent under the formation of the amide coupling. As further elaborated, conventional amino protecting groups are poorly water-soluble thereby impeding amid formation. The solution for increasing the rate of amide formation in an aqueous solvent is to disperse the N-terminal protected amino acids in the aqueous solvent. An aqueous dispersion of protected amino acids is formed by wet pulverizing the protected amino acids to an average particle size in the range of up to 750 nm in the presence of a dispersant. PEG is exemplified as a dispersant. Lower alcohols such as methanol and ethanol as mentioned as useful non-aqueous solvents.

One key aspect of the present invention is the provision of SPPS where the peptide is formed in an aqueous solution but using standard Fmoc α-amine protection strategy. Fmoc is since the mid 1990′ the dominant strategy for the synthetic production of peptides using SPPS (Curr Protoc Protein Sci. 2002 February; CHAPTER: Unit-18.1. doi:10.1002/0471140864.ps1801s26). High quality Fmoc building blocks (amino acids and fragments) are readily available at commercially relevant price points. Many modified derivatives are commercially available as Fmoc building blocks, making synthetic access to a broad range of peptide derivatives straightforward commercially viable.

One objective of the present invention is the reduction of harmful organic solvents in SPPS, specifically Fmoc SPPS.

A further objective is the regeneration of spent solvents emanating from SPPS.

Yet a further objective is to reduce the consumption of solvents in SPPS.

A further objective is the provision of reducing the excess of α-amine protected amino acids and fragments, specifically in Fmoc SPPS while still maintaining a commercially useful primary yield.

A further objective is the provision of a water-based SPPS while applying an amine protection strategy comprising the cleavage of α-amines under alkaline conditions.

A yet further objective is the provision of a water based SPPS while applying an amine proception strategy comprising the implementation of readily available and commercially relevant α-amine protecting groups, specifically Fmoc α-amine protecting groups.

### SUMMARY OF THE INVENTION

The present invention relates to a solid phase peptide synthesis (SPPS) comprising the use of an aqueous solution comprising at least one co-solvent during the removal of the temporary α-amino protecting groups and the subsequent formation of the peptide bond. The invention also encompasses a method for regeneration of the aqueous solution used during the SPPS.

More specifically, the invention relates to a solid-phase peptide synthesis (SPPS) method comprising the provision of: an activated Fmoc-α-amine protected amino acid moiety and an Fmoc-α-amine protected peptide fragment bound to the resin; deprotecting the Fmoc-α-amine protected peptide fragment bound to the resin; coupling of the activated Fmoc-α-amine protected amino acid moiety with the deprotected Fmoc-α-amine protected peptide fragment bound to the resin thereby forming a peptide bond, where the amide (peptide) coupling is performed in an aqueous solution comprising at least one organic co-solvent miscible with water, the aqueous solution capable of sufficiently solubilizing the activated Fmoc-α-amine protected amino acid moiety, and wherein the resin is selected from resins capable of swelling in the presence of the aqueous solution above about 4 mLg⁻¹ (based on weight of the base resin) thereby forming an elongated peptide fragment bound to the resin.

According to an embodiment the invention relates to a solid-phase peptide synthesis method comprising the provision of an activated Fmoc-α-amine protected amino acid moiety and an Fmoc-α-amine protected peptide fragment bound to a resin; deprotecting the Fmoc-α-amine protected peptide fragment bound to the resin; coupling of the activated Fmoc-α-amine protected amino acid moiety with the deprotected Fmoc-α-amine protected peptide fragment bound to the resin thereby forming a peptide bond;
wherein the amide (peptide) coupling is performed in an aqueous solution comprising at least one organic co-solvent miscible with water, the aqueous solution capable of sufficiently solubilizing the Fmoc-α-amine protected amino acid moiety, and wherein the resin is selected from resins capable of swelling in the presence of the aqueous solution above about 4 mL/g−1 (based on weight of the resin) thereby forming an elongated peptide fragment bound to the resin; wherein the activation of the protected amino acid is performed in the presence of a coupling agent and a base, the organic co-solvent having the structural formula:

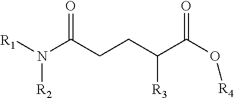

where R₁, R₂, R₃ and R₄ independently selected from alkyls having from 1 to 3 carbon;
the coupling agent being selected from the compounds with the following structural formulas:

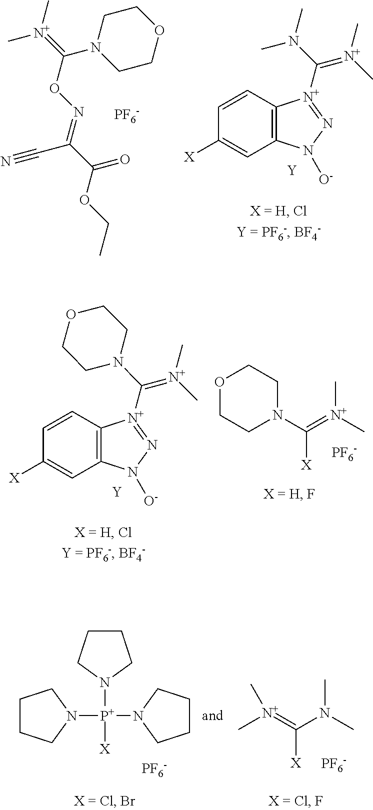

the base being selected from trimethyl derivatives of pyridine; and wherein the resin is selected from copolymers of styrene and ethylene glycol.

Yet a further embodiment of the invention is framed as a solid-phase peptide synthesis method comprising repetitive cycles: each cycle comprising: an activated Fmoc-α-amine protected amino acid moiety and an Fmoc-α-amine protected peptide fragment bound to a resin; deprotecting the Fmoc-α-amine protected peptide fragment bound to the resin;
wherein the amide (peptide) coupling is performed in an aqueous solution comprising at least one organic co-solvent miscible with water, the aqueous solution capable of sufficiently solubilizing the activated Fmoc-α-amine protected amino acid moiety, and wherein the resin is selected from resins capable of swelling in the presence of the aqueous solution above about 4 mLg⁻¹ (based on the weight of resin) thereby elongating the amino acid fragment bound to the resin.

Furthermore, the aqueous reaction solution can be successfully regenerated. Thus, the invention also encompasses a method for the regeneration of aqueous solutions from the SPPS process.

### DESCRIPTION OF THE INVENTION

### General Comments

In the invention Fmoc-α-amine protected amino acid moieties are used. The term Fmoc-α-amine protected amino acid moiety includes Fmoc-α-amine protected natural amino acids, Fmoc-α-amine protected modified natural amino acids, Fmoc-α-amine protected synthetic amino acids and any Fmoc-α-amine protected amino acid fragments. In addition to Fmoc-α-amine protected individual amino acids also fragments can be inserted into the growing peptide residue. An amino acid peptide fragment denotes a compound (peptide) of two or more individual amino acids. When the term Fmoc-α-amine protected amino acid, Fmoc protected amino acid or Fmoc amino acid is used an Fmoc-α-amine protected amino acid moiety is also contemplated if not otherwise stated.

One feature of the present invention is the provision of an aqueous solution comprising at least one organic co-solvent miscible with water, where the aqueous solution sufficiently solubilizes the Fmoc-α-amine protected amino acids, either per se or in their activated forms attained by action of various coupling agents. As the amide (peptide)-bond formation is not thermodynamically favored, the coupling of amino acids to an amino acid or fragment bound to a support usually needs to be conducted in the presence of compounds which creates more thermodynamically favorable reaction conditions and contributes to increased yields. Compounds capable of providing thermodynamically favorable reaction conditions are here referred to as coupling agents. Coupling agents influence the carboxylic acid functionality of the Fmoc protected α-amine amino acid. Depending on conditions, such as pH, the carboxylic acid function may also be provided as the deprotonated carboxylate. The coupling agent may be the only compound involved in providing thermodynamically favorable reaction conditions. However, often the coupling agent interacts with at least a further compound, herein referred to as coupling additives. Some chemistries involving coupling agents and certain amino acids are prone to racemization. The risk of racemization can be reduced or eliminated by the introduction of racemization suppressing additives (coupling additives). Triazoles 1-hydroxy-benzotriazole (HOBt) and 1-hydroxy-7-aza-benzotriazole (HOAt) are commonly employed racemization suppressing additives, especially in combination with carbodiimides such as dicyclohexylcarbodiimide (DCC) and diisopropylcarbodiimide (DIC). As elaborated, the carboxylic acid function of the Fmoc-α-amine protected amino acid or Fmoc-α-amine protected amino acid fragment usually needs to be activated for the provision of useful rates of peptide-bond formation. Depending on the activation chemistry, the activated Fmoc-α-amine protected amino acid can be more or less stable. Should the activated Fmoc-α-amine protected amino acid exhibit sufficient stability the activation of the Fmoc-α-amine protected amino acid can be carried out in the presence of suitable coupling agents in a distinct activation stage. If so, the activated Fmoc-α-amine protected amino acid is transferred to the aqueous solution comprising at least an organic co-solvent.

There are several process alternatives for the activation of Fmoc-α-amine protected amino acids. One possibility is the addition of already activated Fmoc-α-amine protected amino acids to the aqueous solution and subsequent solubilization. An alternative possibility is the activation of Fmoc-α-amine protected amino acids in a suitable solution, such as the aqueous solution of the present invention, and the addition of the activated Fmoc-α-amine protected amino acids in the form of a solution to the resin with the deprotected elongated peptide fragment. A further alternative, which may be useful if the activated Fmoc-α-amine protected amino acids have limited stability, is to activate the Fmoc-α-protected amino acids in-situ with the resin with the deprotected elongated peptide fragment in the aqueous solution comprising at least an organic co-solvent, and a relevant coupling agent. The reaction mechanism from two amino acids to peptide bond usually comprises the formation of intermediates. In the context of the present invention intermediates are any compounds or transient (quasi) compounds formed in the reaction procedure between the Fmoc-α-protected amino acid and the elongated peptide fragment bound to the resin. In the context of the present invention an activated Fmoc-α-protected amino acid can be any one of the intermediates. The term ‘capable of sufficiently solubilizing Fmoc-α-amine protected amino acids and fragments’ encompasses the solubilization of Fmoc-α-amine protected amino acids and/or any intermediates. The aqueous solution disclosed herein solubilizes the Fmoc-α-amine protected amino acids and/or suitable types of activated Fmoc-α-amine protected amino acids.

An Fmoc-α-amine protected amino acid moiety denotes a non-activated Fmoc-α-amine protected amino acid or Fmoc-α-amine protected peptide fragment.

The activated Fmoc-α-amine protected amino acid must be properly solubilized for an individual Fmoc-α-amine protected amino acid to find an amino acid fragment bound to a resin by Brownian motion. By solubilization in the context of this invention is understood any phenomena conducive to the formation of a peptide bond. One phenomenon is the ability of the aqueous solution to provide conditions enabling a useful number of individual activated Fmoc-α-amine protected amino acids to come sufficiently close to the reactive sites of resin-bound peptide fragment.

The successful SPPS is also dependent on the accessibility of the growing resin-bound peptide fragment to reactants. Solvation is a complex concept dependent on a variety of parameters, such as solvent, resin and the type of target peptide. The swelling of a base resin is a useful for the prediction of the accessibility and reactivity of the activated Fmoc-α-amine protected amino acids towards the amino groups on the resin, as well as for efficient deprotections of resin bound Fmoc groups. By base resin it should be understood a resin without a bound peptide (fragment) or optional linker. Base resin may include chemical modifications facilitating the binding of amino acid or linker. In the context of the present invention the swelling is denoted as the volume of swollen resin per weight of resin. The volume of swollen resin per weight resin is generated by a procedure including mixing a determined weight of resin with a solvent and shaking the resin/solvent mixture at room temperature (rt) for 1 hour and thereafter allowing the resin/solvent mixture to stand for 1 hours and then measure the volume of the swollen resin.

In SPPS the target peptide is formed by the stepwise coupling of amine protected amino acids/fragments to a peptide fragment bound to a resin. The term peptide fragment bound to a resin (as used herein) also includes one amino acid bound to a resin. Thus, if one single Fmoc-α-protected amino acid (the 1^{st} amino acid) is bound to a resin possibly with a linker between the Fmoc-α-protected amino acid and the resin the single amino acid bound (linked) to the resin is also encompassed by the term peptide fragment bound to a resin.

The SPPS is a methodology comprising repetitive cycles each cycle comprising at least the coupling of an Fmoc-α-protected amino acid or fragment, and the separation of at least excess reactants from the resin. Before the next cycle commences comprising the addition of the next amine protected amino acid or fragment, the content of the amino acid of fragment of the preceding cycle prone to engage in peptide bond formation (residual amino acids) must be reduced as low as possible for minimizing the formation of non-target peptide variants thereby increasing primary yield (yield prior to post reaction yield-increasing operations such as separation). Residual amino acids from previous cycles can be neutralized by chemical modification transforming residual amino acids to a modified compound not capable of peptide chain elongation (e.g. acetylation of the carboxylic acid into carboxylic acid esters), and neutralized by processes not including chemical modification (e.g. association with a further chemical compound such as complexation). Furthermore, residual amino acids may be neutralized by removal such as filtration, drainage and drainage followed replacement of the drained liquid with displacement liquid free from unwanted compounds specifically amino acids. Often a cycle is followed by drainage and/or washing. Drainage and washing in the field of SPPS do not have an unambiguous meaning. As used herein draining refers to an operation separating the aqueous solution and solutes from non-dissolvable micro-particles specifically resin particles. A washing operation is understood as an operation comprising at least drainage and subsequent addition of an aqueous solution. The method (or cycle) of the invention may comprise draining of the resin. After the draining the aqueous solution of the method of the invention can be added or an alternative solution. If an alternative solution is added to the drained resin, the alternative solution is drained from the resin. Each cycle may contain repetitive drainage inferring multiple additions of alternative solutions or the aqueous solution of the invention. A cycle may contain a drainage stage (where excess reactants are removed) followed by the addition of the aqueous solution of the invention and the addition of new reactant.

According to an aspect of the invention the method may be framed as a solid-phase peptide synthesis method comprising repetitive cycles: each cycle comprising: an activated Fmoc-α-amine protected amino acid or activated Fmoc-α-amine protected peptide fragment and an Fmoc-α-amine protected peptide fragment bound to a resin; deprotecting the Fmoc-α-amine protected peptide fragment bound to the resin; wherein the amide (peptide) coupling is performed in an aqueous solution comprising at least one organic co-solvent miscible with water, the aqueous solution capable of sufficiently solubilizing the activated Fmoc-α-amine protected amino acid or activated Fmoc-α-amine protected peptide fragment, and wherein the resin is selected from resins capable of swelling in the presence of the aqueous solution above about 4 mLg⁻¹ (based on the weight of resin) thereby elongating the amino acid fragment bound to the resin.

Repetitive cycles comprise at least 2 cycles up to any number of cycles necessary for the provision of the target peptide.

In the context of the disclosure by reactants is contemplated amino acids, amino acid fragments to be coupled to the peptide fragment bound to the resin. By reagents is contemplated any other compound not defined as reactants and the reaction solution per se, Examples of reagents include coupling agents, coupling additives, bases but not surfactants. The term peptide fragment bound to a resin includes a peptide fragment covalently bound to the resin either directly to the resin and also a peptide fragment bound to the resin by any type of linker between the peptide fragment and the resin.

An acidic condition is a solution having a pH below 7. Basic conditions are solutions having a pH above 7.

### Embodiments of the Invention

The reaction solution in which the amide bond (peptide bond) is formed is an aqueous solution comprising at least one organic co-solvent miscible with water. By aqueous solution we understand a solution comprising water. Preferably, the aqueous solution comprises at least 50 wt % water, at least 55 wt % water, at least 60 wt % water, at least 65 wt % water, at least 70 wt % water, at least 75 wt % water, at least 80% wt water. The term ‘water’ encompasses any quality ranging from potable tap water to varying qualities of purified water. It is further essential that the aqueous solution comprises at least one organic co-solvent which is miscible in water, at least miscible at the intended co-solvent water ratio(s). By water miscibility herein is meant the ability of two or more liquids (solvents) to mix with each other to form a homogeneous solution. The organic co-solvent may be fully (or totally) miscible with water, that is the organic co-solvent is miscible at any co-solvent/water ratio (weight of the co-solvent to weight of final solution). If the organic co-solvent is totally miscible with water the miscibility of the co-solvent is 100% (wt co-solvent to wt of final solution). The co-solvent may not be totally miscible with water. If the co-solvent is not totally miscible with water than a ratio co-solvent and water is applied for the co-solvent to be fully miscible in water. It is sufficient that the organic co-solvent is miscible with water to the extent to be able to form a homogeneous solution, even if the co-solvent does not exhibit 100% miscibility with water.

According to an aspect the amide coupling is performed in the absence of any dispersants.

According to a further aspect, the activated Fmoc-α-amine protected amino acid moieties are not dispersed or provided in form of a dispersion. More specifically, the Fmoc-α-amine protected amino acid moieties are not subjected to particulation, such as pulverization

According to a further aspect the (activated) Fmoc-α-amine protected amino acid moieties are solubilized in the aqueous solution comprising at least one organic co-solvent signifying that individual (activated) Fmoc-α-amine protected amino acid moieties are dissolved, i.e. that individual (activated) Fmoc-α-amine protected amino acid moieties are solvated or surrounded by a layer of solvent molecules (water and co-solvent). The size of individual (activated) Fmoc-α-amine protected amino acid moieties are governed by the size of the respective amino acid moiety in combination with the size of the Fmoc group. The size of individual (activated) Fmoc-α-amine protected amino acid moieties is generally below about 5 nm. Most non-protected naturally occurring amino acids have a size below 1 nm.

According to an aspect of the invention the solubility of the reactants (Fmoc-α-protected amino acids and fragments) may be enhanced by the presence of surface-active compounds (surfactants).

There is a delicate interplay of several parameters for the successful commercial application of SPPS. One requirement of SPPS is that the α-amine function of the amino acids or amino acid fragments used for elongating the growing peptide fragment bound to the support (resin) are protected, otherwise it is impossible to form the target peptide. The α-amine function of the amino acids or amino acid fragments deployed in the present invention are protected by Fmoc groups. The Fmoc-moiety (fluorenylmethyloxycarbonyl) is a tricyclic aromatic carbamate increasing the hydrophobicity of the Fmoc protected amino acid or amino acid fragment. It is important for the provision of a useful reaction rate that activated Fmoc-α-amine protected amino acid or activated Fmoc-α-amine protected peptide fragment is sufficiently solubilized. By Fmoc-α-amine protected amino acid or Fmoc-α-amine protected peptide fragment in the context of solubilization is meant any non-activated Fmoc-α-amine protected amino acid or any transient (activated) form of the Fmoc-α-amine protected amino acid until formation of the peptide bond between the Fmoc-α-amine protected amino acid and the growing peptide fragment bound to the resin.

Usually, the carboxylic acid function or carboxylate anion of the Fmoc-α-amine protected amino acid or Fmoc-α-amine protected peptide fragment is activated by suitable activation chemistries including one or several activating compounds (herein also referred to as coupling agents, CAs).

In the context of the present invention solubilization is the capacity of the aqueous solution to successfully transport individual activated Fmoc-α-amine protected amino acids sufficiently close to the reaction site of the growing peptide fragment bound to the support and preferably that the rate limiting step (of the peptide bond formation) is predominantly governed by the rate of the chemical reaction and not by diffusion/Brownian motion.

For reasons elaborated above Fmoc protected amino acids are difficult to solubilize in aqueous solutions. Organic aprotic polar solvents, specifically DMF, are the solvents of choice in SPPS using Fmoc-α-amine protected amino acids, one reason being the ability to solubilize Fmoc-α-amine protected amino acids. By the presence of an organic co-solvent in the aqueous solution, sufficient solubilization of Fmoc-α-amine protected amino acids or their activated forms is obtained.

A further important parameter for the commercially successful application of SPPS is the swelling of the resin. The resin applied in the present invention is selected from base resins that swell at least 4 mLg⁻¹ in the chosen aqueous solution comprising at least one organic co-solvent miscible in water. A more detailed disclosure for quantifying the swelling reference is made in the experimental part. According to an aspect the resin has the capacity to exhibit swelling in the range from about 4 mLg⁻¹ up to about 8 mLg⁻¹.

The protective Fmoc group is base-labile, thus, the Fmoc groups is cleavable under basic conditions. Since Fmoc is base-labile, reactive amino acid side chains are typically protected by acid-labile protection groups (such as tert-butyl). Thus, chain elongation is usually carried out under neutral to alkaline condition. When the target peptide bound to the resin has been formed the target peptide is cleaved from the resin. Typically, the cleavage of the peptide is conducted under acidic conditions. According to a further aspect the resin has also the characteristics of not swelling excessively during cleavage of the crude target peptide from the resin. Thus, the base resin used in the method should typically has the ability to swell at least about 4 mLg⁻¹ while not swelling excessively during the acid conditions during cleavage from the resin. The swelling is to an extent correlated to the solvent used for cleavage. Usually, a rather strong acid is used for cleavage.

According to an aspect, the base resin is characterized as being capable of swelling in the presence of the aqueous solution above about 4 mLg⁻¹ and below about 12 mLg⁻¹, suitably below about 10 mLg⁻¹ under the conditions for peptide resin cleavage.

An embodiment of the invention is that the amide-coupling is performed in the presence of at least an activation/coupling agent (CA). According to an aspect of the invention the coupling agent is selected from the group consisting of carbodiimides, optionally in the presence of N-hydroxylamine-based coupling additives, uronium (amidium) based CAs, phosphonium based CAs, compounds converting acids to acid chlorides, compounds converting carboxylic acids to the corresponding acyl fluorides, and triazine-based compounds.

According to a further aspect of the invention the coupling agent is selected from the group consisting of diisopropylcarbodiimide (DIC), dicyclohexylcarbodiimide (DCC), 1-Ethyl-3-(3-dimethylaminopropyl)carbodiimide (EDC×HCl) (optionally in the presence of coupling additives such as 1-Hydroxybenzotriazole (HOBt), 6-Chloro-1-hydroxybenzotriazole (Cl-HOBt), 1-hydroxy-7-aza-benzotriazole (HOAt), 2-Hydroxypyridine-N-oxide (HOPO), ethyl cyanohydroxyiminoacetate (Oxyma), Oxyma-B, N-Hydroxysuccinimide (HOSu), N-Hydroxy-5-norbornene-2,3-dicarboxylic acid imide (HONB), Hexafluorophosphate benzotriazole tetramethyl uronium (HBTU), O-(Benzotriazol-1-yl)-N,N,N′,N′-tetramethyluronium tetrafluoroborate [O-[N,N,N′,N′-Tetramethyl-O-(1H-benzotriazol-1-yl)uronium hexafluorophosphate] (TBTU), O-[(Ethoxycarbonyl)cyanomethylenamino]-N,N,N′,N′-tetramethyluronium hexafluorophosphate (HOTU), O-[(Ethoxycarbonyl)cyanomethylenamino]-N,N,N′,N′-tetramethyluronium tetrafluoroborate (TOTU), O-(1H-6-chlorobenzotriazole-1-yl)-1,1,3,3-tetramethyluronium hexafluorophosphate (HCTU), O-(6-Chlorobenzotriazol-1-yl)-N,N,N′,N′-tetramethyluronium tetrafluoroborate (TCTU), 1-[Bis(dimethylamino)methylene]-1H-1,2,3-triazolo[4,5-b]pyridinium 3-oxide hexafluorophosphate, hexafluorophosphate azabenzotriazole tetramethyl uronium (HATU), O-(7-Azabenzotriazole-1-yl)-N,N,N′,N′-tetramethyluronium tetrafluoroborate (TATU), 1-cyano-2-ethoxy-2-oxoethylidenaminooxy)dimethylamino-morpholino-carbenium hexafluorophosphate (COMU), 1-[(Dimethylamino)(morpholino)methylene]-1H-[1,2,3]triazolo[4,5-b]pyridine-1-ium 3-oxide hexafluorophosphate (HDMA), HDMB, 6-chloro-1-((dimethylamino)(morpholino)-methylene)-1H-benzotriazolium (HDMC), Benzotriazol-1-yloxytris(dimethylamino)phosphonium hexafluorophosphateC (BOP), (Benzotriazol-1-yloxy)tripyrrolidinophosphonium hexafluorophosphate (PyBOP), [Ethyl cyano(hydroxyimino)acetato-O²]tri-1-pyrrolidinylphosphonium hexafluorophosphate (PyOxim), 6-Chloro-benzotriazole-1-yloxy-tris-pyrrolidinophosphonium hexafluorophosphate (PyClock), N,N,N′,N′-Tetramethylchloroformamidinium Hexafluorophosphate (TCFH), N-(chloro(morpholino)methylene)-N-methylmethanaminium hexafluorophosphate (DMCH), Chlorotripyrrolidinophosphonium hexafluorophosphate (Pyclop), tetramethylfluoroformamidinium hexafluorophosphate (TFFH), tetramethylammonium trifluoromethanethiolate ((Me₄N)SCF₃), 2-Chloro-4,6-dimethoxy-1,3,5-triazine (CDMT), 2,4-Dichloro-6-methoxy-1,3,5-triazine (DCMT), 4-(4,6-Dimethoxy-1,3,5-triazin-2-yl)-4-methylmorpholinium chloride (DMTMMCI), 4-(4,6-Dimethoxy-1,3,5-triazin-2-yl)-4-methylmorpholinium tetrafluoroborate (DMTMMBF₄) and 2-(4,6-dimethoxy-1,3,5-triazinyl)trialkylammonium salts (DMT-Ams).

According to yet a further aspect, the coupling agent is selected from compounds from the group consisting of COMU, HDCM, 6-chloro-1-((dimethylamino)(morpholino)-methylene)-1H-benzotriazolium hexafluorophosphate 3-oxide (HDMC), Chloro-N,N,N′,N′-tetramethylformamidinium hexafluorophosphate (TCFH), and tetramethylfluoroformamidinium hexafluorophosphate (TFFH).

Yet another aspect is to select the coupling agent from the group consisting of the compounds with the following structural formulas:

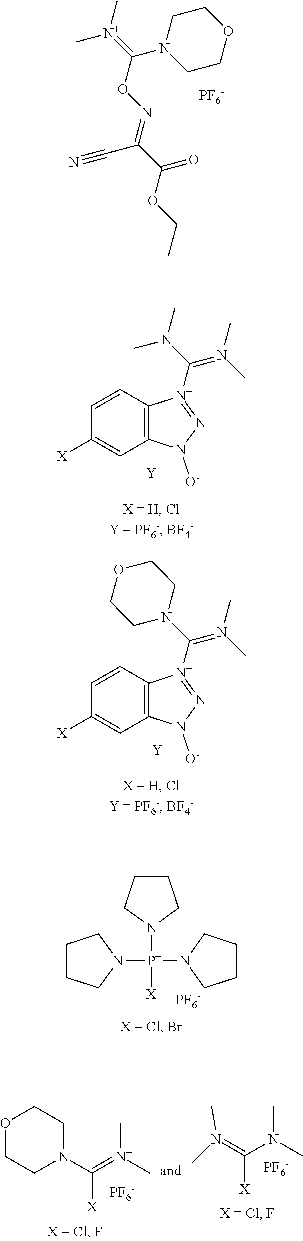

A further aspect of the invention is to select coupling agents from compounds of:

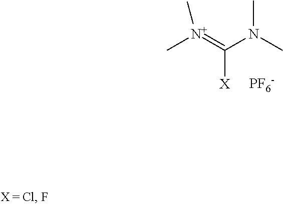

### Co-Solvents

Any organic co-solvents miscible with water fulfilling the criteria of the method, i.e. sufficiently solubilize Fmoc-α-amine protected amino acids as such or in their activated forms while also swelling the resin of at least 4 mLg⁻¹, can be envisaged for use as co-solvent. The aqueous solution may also comprise any number and ratios of co-solvents. Dipolar aprotic co-solvents are particularly preferred, specifically dipolar aprotic co-solvents that solubilize Fmoc substituted amino acids well.

According to an aspect, the co-solvent has a polarity from about 0.2 up to about 0.5. The lower end of the polarity of the co-solvent can be about 0.21, about 0.22, about 0.23, about 0.24, about 0.25, about 0.26, about 0.27, about 0.28, about 0.29, about 0.30. The upper end of the polarity of the co-solvent can be about 0.49, about 0.48, about 0.47, about 0.46, about 0.45, about 0.44, about 0.43, about 0.42, about 0.42, about 0.40. Any lower and upper level of polarity may be combined.

According to an aspect the co-solvent is an organic co-solvent miscible in water, preferably an organic polar aprotic co-solvent, further characterized by having a polarity in any range given by any combination of upper or lower polarity values disclosed herein.

Preferred dipolar aprotic co-solvents are the following: N-methylpyrrolidone (NMP), N-ethylpyrrolidone (NEP), N-propylpyrrolidone (NPP), N-butylpyrrolidone (NBP), N-pentylpyrrolidone (NPeP), N-hexylpyrrolidone (NHP), N-heptylpyrrolidone (NHeP), N-octylpyrrolidone (NOP), dimethylformamide (DMF), diethylformamide (DEF), dipropylformamide (DPF), N-formylpyrrolidine (NFP), N-formylmorpholine (NFM), N-methylcaprolactam (MCL), 1,3-dimethyl-2-imidazolidinone (DMI), 1,3-dimethyl-3,4,5,6-tetrahydro-2-pyrimidinone (DMPU), dimethylsulfoxide (DMSO), diethylsulfoxide (DESO), sulfolane, (1R)-7,8-dioxabicyclo[3.2.1]octan-2-one (dihydrolevoglucosenone, cyrene®), N,N-dimethylacetamide (DMA), N,N,N′,N′-tetraethyl sulfamide (TES), 1-butyl-3-methylimidazolium chloride (BMIMCI), 1-butyl-3-methylimidazolium bromide (BMIMBr), 1-butyl-3-methylimidazolium iodide (BMIMI), 1-butyl-3-methylimidazolium tetrafluoroborate (BMIMBF₄), methyl-5-(dimethylamino)-2-methyl-5-oxopentanoate either in its neat (pure) form or as the dominant component of the mixture of compounds known as RhodiaSolv® PolarClean (PC).

According to an aspect of the invention, the organic co-solvent is selected from co-solvents having the chemical structure (1)

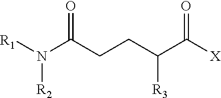

where R1, R2, and R3 are independently selected from alkyls having from 1 to 3 carbon atoms, with the proviso that if X is an oxygen atom, then one alkyl group comprising from 1 to 3 carbon atoms is bound to the oxygen atom; or with the provision that if X is a nitrogen atom, then two alkyl groups are bound to the nitrogen atoms; or mixtures thereof, the alkyl group(s) independently selected from 1 to 3 carbon atoms.

According to a further aspect, the organic co-solvent is selected from co-solvents having the chemical structure (2)

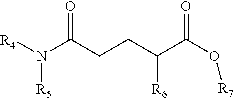

where R₄, R₅, R₆ and R₇ independently are selected from alkyls having from 1 to 3 carbon atoms. According to an aspect R₄, R₅, R₆ and R₇ of structure (2) are methyl.

The co-solvent may be present in the aqueous solution in any ratio, preferably between 10 to 90% v/v based on the total volume of solution, preferably between 20 to 50% v/v.

According to an aspect the co-solvent comprising methyl-5-(dimethylamino)-2-methyl-5-oxopentanoate. Methyl-5-(dimethylamino)-2-methyl-5-oxopentanoate is the main constituent of the commercially available solvent PolarClean® or Rhodiasolv PolarClean®. The co-slovent comprising methyl-5-(dimethylamino)-2-methyl-5-oxopentanoate may be formed by a process starting from 2-methylglutaronitrile (which is a by-product during hydrocyanation of butadiene). 2-Methylglutaronitrile undergoes hydrolysis to produce 2-methylglutaric acid which is cyclized to form 2-methylglutaric anhydride. The esterification and amidation of the two carbonyl groups in different orders ultimately lead to the formation of predominantly methyl-5-(dimethylamino)-2-methyl-5-oxopentanoate but also the regioisomer of methyl 4-(dimethylamino)-2-ethyl-4-oxobutanoate and to a lesser extent 2-N,N,N′,N′-pentamethylglutaramide and dimethyl 2-methylglutarate.

PolarClean® contains about 80-90 wt % methyl-5-(dimethylamino)-2-methyl-5-oxopentanoate, 6-12 wt % regioisomer of methyl 4-(dimethylamino)-2-ethyl-4-oxobutanoate, 3-7 wt % 2-N,N,N′,N′-pentamethylglutaramide and 0.5-3 wt % of dimethyl 2-methylglutarate.

According to a further aspect the co-solvent comprises 80-90 wt % methyl-5-(dimethylamino)-2-methyl-5-oxopentanoate, 6-12 wt % regioisomer of methyl 4-(dimethylamino)-2-ethyl-4-oxobutanoate, 3-7 wt % 2-N,N,N′,N′-pentamethylglutaramide and 0.5-3 wt % of dimethyl 2-methylglutarate.

### Coupling Agents

Any of the process aids deemed as coupling agents for amide bond forming reactions (see for example Chem. Rev. 2011, 111, 6557-6602) can be envisaged for use in the aqueous SPPS of the invention. More specifically with regards to manufacturing of peptides on large scales, use of peptide coupling reagents suitable for large scale applications (see for example Org. Process Res. Dev. 2016, 20, 140-177) and having suitable thermal stability profiles (see for example Org. Process Res. Dev. 2018, 22, 1262-1275) is particularly advantageous. Additionally, peptide coupling reagents that have been shown to work adequately in aqueous solutions (see for example Tetrahedron Lett. 2017, 58, 4391-4394) are particularly advantageous for use in the present aqueous SPPS. The said peptide coupling agents may include, but are not limited to carbodiimides such as diisopropylcarbodiimide (DIC), dicyclohexylcarbodiimide (DCC) and 1-Ethyl-3-(3-dimethylaminopropyl) carbodiimide (EDC)×HCl either as such or in the presence of the so-called coupling additives for example based on the N-hydroxylamine based compounds such as triazoles 1-hydroxy-benzotriazole (HOBt), Cl-HOBt, 1-hydroxy-7-aza-benzotriazole (HOAt), 2-Hydroxypyridine-N-oxide (HOPO), ethyl cyanohydroxyiminoacetate (Oxyma), 5-(Hydroxyimino)1,3-dimethylpyrimidine-2,4,6-(1H,3H,5H)-trione (Oxyma-B), HOSu and N-hydroxy-5-norbornene-endo-2,3-dicarboxyimide (HONB). Additionally, any of the so-called uronium (amidinum) coupling agents can be used in aqueous SPPS as well, for example HBTU, TBTU, HOTU, TOTU, HCTU, TCTU, HATU, TATU, COMU, HDMA, HDMB, HDMC. Furthermore, phosphonium reagents such as BOP, PyBOP, PyOxim and PyClock can be used as well. Further, any of the reagents converting acids to acid chlorides (see for example Tetrahedron 2015, 71, 2785-2832) can be utilized as well for example TCFH, DMCH and PyCloP. Further, process aids converting carboxylic acid to the corresponding acyl fluorides such as TFFH and tetramethylammonium trifluoromethanethiolate ((Me₄N)SCF₃) (Org. Lett. 2017, 19, 5740-5743) can be used in the present aqueous SPPS as well. Moreover, triazine based coupling reagents such as CDMT, DMTMMCI, DMTMMBF₄ and DMT-Ams (Molecules, 2021, 26, 191) can be used as well. The person skilled in the art will know that the said coupling agents can be used in combination with any other coupling agent to enhance the rate or chemoselectivity of the peptide bond formation in the context of the aqueous SPPS of the present invention. Furthermore, a person skilled in the art will also know that the said coupling agents can be used in the context of aqueous Fmoc/t-Bu SPPS not only by converting Fmoc α-amino substituted amino acids to their activated counterparts capable of reacting with the amino terminus of the growing peptide chain in-situ (i.e. in the SPPS), but also in a separate vessel in which the activated species is premade in a suitable solution and either used as such in the context of aqueous Fmoc/t-Bu SPPS or, the activated α-amino substituted species can be isolated and the said activated species are then added to the SPPS reactor, either as such or in a suitable solution of water with appropriate cosolvent(s).

### Coupling Additives

Coupling additives are any reagents used together with coupling agents with the purpose of increasing rate of amide bond formation, chemoselectivity of amide bond formation, or both. Numerous standard couplings additives can be envisaged in the current invention (see for example Chem. Rev. 2011, 111, 6557-6602), specifically the hydroxylamine-based coupling additives such as HOBt, Cl-HOBt, HOAt, 2-Hydroxypyridine-N-oxide (HOPO), Oxyma, Oxyma-B, HOSu and HONB or the 2-mercaptobenzothiazole (2-MBT) can be used.

### Coupling Base

Furthermore, a person skilled in the art will know that the above noted means of converting Fmoc-α-amino substituted amino acids to their activated counterparts can take place either in the absence of any base, or in a presence of any organic or inorganic base or a combination thereof which can have a beneficial effect on rates and/or chemoselectivities of amide bond forming reactions in the context of aqueous Fmoc/t-Bu SPPS.

Thus, according to a further embodiment the amide coupling is performed in the presence of a base.

According to an aspect the base (coupling base) is selected form alkyl derivatives of pyridine. More specifically, the based is selected from alkyl, dialkyl and trialkyl derivatives of pyridine, the alkyl comprising from 1 to 3 carbon atoms, preferably the alkyl comprises 1 or 2 carbon atoms. Preferably, the alkyl is methyl. The base may be selected from methyl derivatives of pyridine. The base may be selected from picoline, lutidine and collidine and any regioisomers thereof.

According to a further aspect, the base is selected from collidine and any regioisomers thereof.

The said bases may include, but are not limited to, aliphatic amines such diisopropylethylamine (DIEA) and N-methylmorpholine (NMM), aromatic amines such as pyridine, picoline (2-methylpyridine or any regioisomer thereof) lutidine (2,6-dimethylpyridine or any regioisomer thereof), collidine (2,4,6-trimethylpyridine or any regioisomer thereof), imidazole or N-methylimidazole (NMI) and inorganic bases such as for example any phosphate, carbonate, sulfate, acetate, borate for example in their lithium, sodium, potassium, calcium or tetraalkylammonium forms. A person skilled in the art will know that the aforementioned coupling agents and bases may be combined with Fmoc α-amino substituted amino acids in any ratios and equivalency and at any temperature with view on attaining suitable rates and chemoselectivities in the context of aqueous Fmoc/t-Bu SPPS. The reaction times for the said amide bond forming processes can be from seconds to 16 hours, more preferably from 10 min to 2 hours. If needed, the amide bond forming step can be repeated (so-called recoupling) and if deemed appropriate the amide bond forming reaction can be terminated by carrying out a so-called capping reaction, in which any remaining free amino groups on the insoluble polymer bound peptide resin is capped by a suitable capping agent for example in the form of a suitable organic acid anhydride such as acetic anhydride or suitable organic acid such as acetic acid or phenylacetic acid in the presence of any suitable coupling agents.

### Surface Active Agents, Surfactants

According to an embodiment of the invention, surface active compounds (surfactants) may be present in the aqueous solution during the formation of the peptide bond or during the removal of the temporary α-amino protecting group. Useful surfactants include nonionic surface active compounds comprising PEG such as surfactants based on polyethylene glycol p-(1,1,3,3-tetramethylbutyl)-phenyl ether (triton-x), derived from (2R)-2,5,7,8-tetramethyl-2-[(4R,8R)-4,8,12-trimethyltridecyl]-3,4-dihydro-2H-1-benzopyran-6-ol (α-tocopherol) or poly(oxy-1,2-ethanediyl), α-[[(2S)-1-(1-oxododecyl)-2-pyrrolidinyl]carbonyl]-ω-methoxy-(PS-750-M). The surfactant may be present in the aqueous solution in an amount of less than 10% v/v based on the amount of water used. preferably at <5% v/v based on the amount of water used.

### Resin

One objective with the invention is the reduction of organic solvents, especially hazardous organic solvent. The measure for reducing organic solvents is the introduction of aqueous solutions comprising an organic co-solvent. According to the invention the selected resin has a swelling of at least 4 mLg⁻¹ in the aqueous solution. According to an aspect the resin has a swelling of at least 4 mLg⁻¹ but below 10 mLg⁻¹ in the aqueous solution used for e.g. cleaving the peptide from the resin.

A wide variety of resins can be implements as long as they provide a swelling characteristic as disclosed herein, i.e. above about 4 ml/g in the chosen aqueous solution.

According to an aspect the resin is selected from styrene-based resins, amide-based resins such as polyacrylamide-based resins (i.e. polymers comprising amide groups), polyethylene based resins, acrylate based resins, acrylate ethoxylate based resins, amino acid based resins and ethylene amide based resins, polyester-based resins, polyether-based resins, acrylamide-based resins.

Polyacrylamide based resins also include resins comprising polymerized amino acids comprising at least three binding sites. Such amino acid-based resins may comprise lysine analogues such as any one of diaminopropionic acid, diaminobutyric acid and diaminopentanoic acid, diaminohexanoic acid.

According to yet another aspect the resin comprises hydrophilic residues, suitably the resin comprises hydrophilic residues at a ratio enabling the resin to swell above about 4 ml/g in the aqueous solutions disclosed herein. A suitable hydrophobic monomer or oligomer are alkylene glycol and polyalkylene glycol such as ethylene glycol and propylene glycol and polyethylene glycol and polypropylene glycol. The hydrophilic residue (monomer(oligomer) is present in a resin comprising hydrophilic residues at a ratio of above about 30% based on total weight of resin, above about 40, above about 45%, above about 50%, above about 60%, above about 65%. Some resins comprise hydrophilic residues at a ration above about 95% wt, above about 99% wt. ChemMatrix® is an example of a polyethylene glycol resin comprising above 99% wt of PEG.

Often the resins comprise a core polymeric matrix, which may be chemically modified by suitable oligomers and/or polymers (e.g. PEG), and linkers. E.g. a polystyrene based resin (and any other of the mentioned types of resins) should be construed as a resin comprising a core polymeric matrix essentially comprising polystyrene.

According to a further aspect the core or resin matrix of any of the herein disclosed resin types are modified by hydrophobic moieties such as hydrophilic monomers, oligomers and/or polymers. Exemplified hydrophilic moieties include polyethylene glycol (PEG).

According to yet a further aspect the polymeric matrix of any of the resin types disclosed herein are cross-linked. E.g. a polystyrene based resin is preferably cross-linked with divinylbenzene (DVB) at an amount of from 0.2 up to 5 mol % DVB.

According to yet a further aspect the resin comprises PEG. The PEG, present as oligomers and/or polymers may be grafted on a polymer matrix. The PEG moieties may comprise a few PEG monomers/units up to several hundred monomers having a molecule weight form about 250 g/mol up to about 10000 g/mol, typically form about 300 g/mol up to about 5000 g/mol.

According to a further aspect the resin is selected from copolymers comprising a cross-linked polystyrene matrix and polyethylene glycol. Suitably, the PEG is grafted to the matrix preferably vis an ethyl ether group.

Amino acid based resins include poly-3-lysine based resins suitably cross-linked with a dicarboxylic acid, e.g. sebacic acid.

Polyethylene based resins include resins comprising a polymer matrix consisting essentially of PEG (PEG-units/monomers) such as ChemMatrix® and NovaPEG.

Resins comprising PEG may comprise a polymer matrix based on polystyrene, polyacrylamide, or ethylene amide. Exemplified resins comprising PEG are NovaSyn® TG resins, PEGA resins, and cross-linked ethoxylate acrylate resin (CLEAR) resins.According to an aspect the resin is selected from polymers comprising polyethylene glycol (PEG) and polymers obtained by polymerization of trimethylolpropane ethoxylate triacrylate.

According to yet a further aspect the resin is selected from polymers comprising polyethylene glycol (PEG). The resin is suitably a co-polymer comprising polyethylene glycol (PEG).

According to a further aspect the resin is selected from co-polymers comprising poly styrene (PS) and polyethylene glycol (PEG). A suitable resin may also be defined as a polymer in particulate form comprising polystyrene and polyethylene glycol.

According to an aspect the resin is a resin comprising polystyrene and PEG wherein the PEG is present at a ratio of above about 40% based on total weight of resin, above about 45%, above about 50%, above about 60%, above about 65%. The resin comprising polystyrene and PEG has a PEG ration of below about 95% based on total weight of resin, suitably below about 90%, below about 85% based on total weight of resin.

According to a further aspect the resin is selected from crosslinked polystyrene comprising polyethylene glycol (PEG) which is grafted on the polystyrene. These resins may also be denoted polystyrene (PS) polyethylene glycol (PEG) graft co-polymers.

The polystyrene (PS) polyethylene glycol (PEG) graft co-polymer has typically a matrix or core of crosslinked polystyrene. The PS matrix may be modified thereby introducing PEG moieties, e.g. grafting of PEG to the PS matrix, by anionic graft copolymerization such as anionic graft polymerization of ethylene oxide on tetraethylene glycol, referred to as POE-PS, or by coupling Nω-Boc or Fmoc-polyethylene glycol acid or polyethylene glycol diacid to amino functionalized polystyrene (PEG:PS). The resulting PS-PEG copolymer comprises a PS-matrix on which PEG is grafted/linked.

The PEG is suitably attached to the polystyrene backbone via a linker. Such linker may be selected from alkyls and/or benzyl ethers.

The resin matrix such as PS is suitably grafted with PEG having an average molecular weight in the range of from about 1000 to about 5000 Dalton, from about 1500 up to about 4500 Dalton, from about 2000 up to about 4000 Dalton. The resin matrix such as PS-PG graft co-polymer may comprise PEG in an amount of from 40 to 80% (w/w), suitably from 50 to 70%.

The peptide fragment bound to the resin encompassed the peptide fragment covalently bound to the resin by a linker. It is preferred that the linker is acid-labile, inferring that the linker facilitates the cleavage of the peptide fragment under acid conditions.

A wide variety of linkers can be used for aqueous Fmoc (t-Bu) SPPS including the most commonly used linkers such as Rink, Rink amide, Ramage, Sieber, 2-chlorotrityl chloride, 4-methylbenzhydryl, Wang, hydroxymethylbenzoyl (HMBA), hydroxylmethylphenoxyacetyl (HMPA) and hydrazinobenzoyl (HZB). A person skilled in the art will know that any linker routinely used for Fmoc/t-Bu SPPS (see e.g. Chem. Rev. 2000, 100, 2091-2158) can be used for the aqueous Fmoc/SPPS disclosed herein.

### Cleavage of Crude Peptide from Resin

An aspect of Fmoc/t-Bu SPPS is the detachment of a target peptide from the insoluble polymer support. Depending on the synthetic strategies used in the context of synthesis of a given target molecule, this detachment of the peptide from the support may or may not be accompanied by the removal of the amino acid side chain protecting groups. As the final peptide resins obtained from conventional Fmoc/t-Bu SPPS and aqueous Fmoc/t-Bu SPPS respectively are of comparable attributes any methods or protocols used for the detachment of peptide from insoluble support in standard Fmoc/t-Bu SPPS (J. Pept. Sci. 2016, 22, 4) can be used in the context of the aqueous SPPS disclosed herein. Typically, a peptide resin from aqueous Fmoc/t-Bu SPPS upon completion of a synthesis sequence is thoroughly washed using an innocuous organic solvent (e.g. alcohol such i-PrOH, ether such as diethylether or alkane such heptane or petroleum ether), dried to constant weigh in vacuo upon which the peptide resin thus obtained is cleaved with a suitable organic acid such as trifluoroacetic acid (TFA) in the presence of various process aids (so called scavengers) used to intercept any reactive species formed during a cleavage. Subsequently, the target crude peptides are isolated for example by means of precipitation using suitable antisolvents such as for example Et₂O, i-Pr₂O, tert-Butyl methyl ether (MTBE), cyclopentyl methyl ether (CPME), 2-methyltetrahydrofuran (2-MeTHF), 4-methyltetrahydropyran (4-MeTHP), EtOAc, hexane, heptane, petroleum ether(s) or any combination thereof. Alternatively, the target crude peptides can be isolated by suitably neutralizing the target peptide containing an acid cleavage solution, for example using an aqueous solution of a suitable salt such as for example ammonium acetate or ammonium formate of a suitable strength to which suitable water miscible organic cosolvents such as acetonitrile (MeCN), EtOH or isopropyl alcohol (i-PrOH) may be added as deemed appropriate from the standpoint of physicochemical attributes of a given target peptide.

### Fmoc Deprotection

Repetitive, high yielding and highly chemoselective removals of Fmoc α-amino protecting groups is an important aspect of any successful Fmoc/t-Bu SPPS methodology (J. Pept. Sci. 2016, 22, 4). The said Fmoc removals can be effectuated by a wide variety of, typically basic, process aids and in the context of aqueous Fmoc/t-Bu SPPS any of these process aids or combinations thereof can be used.

According to an aspect the Fmoc groups is cleaved by the application of Fmoc removal compounds selected from secondary cyclic amines and secondary non-cyclic amines.

The Fmoc removal compounds may also be selected from monocyclic secondary amines and more particularly from monocyclic secondary amines where the nitrogen atom forms part of the cycling ring structure.

The Fmoc removal compounds may also be selected from piperidine, 4-methylpiperidine, 3-methylpiperidine, 2-methylpiperidine, morpholine, piperazine, pyrrolidine.

These process aids may include, but are not limited to, piperidine, 4-methylpiperidine, 3-methylpiperidine, 2-methylpiperidine, morpholine, piperazine, pyrrolidine, diethylamine, 1,8-Diazabicyclo[5.4.0]undec-7-ene (DBU), tetrabutylammonium fluoride (TBAF), ethanolamine, ammonia, lithium hydroxide, sodium hydroxide and potassium hydroxide. Further useful bases include 4-(aminomethyl)piperidine (4-AMP) and tris(2-aminoethyl)amine (TAEA) 2-(aminoethoxy)ethanol (AEE). A person skilled in the art will know that the aforementioned Fmoc deprotection agents can be used or combined in any equivalency compared to the molar amount of growing peptide on the insoluble polymer support, and the person skilled in the art will also know that the Fmoc deprotection agent(s) can be added all at once at the outset, or throughout the course of an Fmoc deprotection as deemed appropriate with view on rates and chemoselectivities of the Fmoc deprotection reactions and that the said Fmoc deprotections can be carried out at any temperature as viewed appropriate from the standpoint of Fmoc deprotection conversion and chemoselectivity. The reaction times of the said deprotection can vary from seconds to about 60 minutes, preferably from about 1 minutes to about 30 minutes Further in particular with regard to chemoselectivity of the said Fmoc deprotections, any method or approach commonly used to suppress side reactions typically occurring throughout the course of an Fmoc deprotection reaction can be used in the context of aqueous Fmoc/t-Bu SPPS. For example, the occurrence of widely common side reactions in Fmoc/t-Bu SPPS centered in the formation of aspartimide related impurities (see e.g. Tetrahedron 2011, 67, 8595-8606) can be suppressed for example by addition of acid agents such as HOBt, Oxyma or formic acid (Org. Lett. 2012, 14, 5218-5221), by use of a weaker base such as piperazine as the process aid for Fmoc deprotection (Lett. Pept. Sci., 2000, 7, 107-112, RSC Adv., 2015, 5, 104417-104425), by use of adequately side chain protected Fmoc-Asp(X)-OH derivative such as Fmoc-Asp(OBno)-OH (J. Pept. Sci. 2015, 21, 680-687) or (S,Z)-4-((((9H-Fluoren-9-yl)methoxy)carbonyl)amino)-4-carboxy-1-cyano-1-(dimethylsulfonio) but-1-en-2-olate (Fmoc-Asp (CSY)-OH) (Nature Comm. 2020, 11, 982) or by using a suitable Fmoc-Asp(OtBu)-OH containing, backbone protected dipeptide such as Fmoc-Asp(OtBu)-(Dmb)AA-OH or Fmoc-Asp(OtBu)-(Hmb)AA-OH. Furthermore, a person skilled in the art will know that the removal of the Fmoc group can be aided by a suitable process aid that will scavenge the dibenzofulvene (DBF) liberated during the course of an Fmoc deprotection reaction, and that useful agents that are efficient in scavenging DBF liberated during Fmoc deprotection reaction are typically thiols such as DTT, thiophenol, thiosalicylic acid (Tetrahedron Lett. 2000, 41, 5329), 1-octanethiol (J. Peptide Sci. 2002, 8, 529-542), DODT, N-acetyl cysteine, mercaptopropionic acid (Angew. Chem. Int. Ed. 2017, 56, 7803-7807) or thiomalic acid (Angew. Chem. Int. Ed. 2021, 60, 7786-7795).

### Washes

As elaborated herein, the SPPS comprises repetitive cycles, the cycles comprising washing stages. An aspect of any Fmoc/t-Bu SPPS methodology is how washes between Fmoc deprotections and couplings as well as washes between couplings and Fmoc deprotections are carried out, as the vast majority of solvent consumption during Fmoc/t-Bu SPPS occurs during the aforementioned washing steps. One advantage of aqueous Fmoc/t-SPPS is that the hazardous solvent typically used as the washing medium (for example DMF and NMP) is replaced with benign and inexpensive water, which is used in combination with appropriate cosolvent(s) as specified above. The person skilled in the art will know that the water/cosolvent(s) ratios used in washes may vary from the water/cosolvent(s) ratios used in the couplings and Fmoc removals respectively in order to optimize the washing efficiency as well as minimize the cost of washing, and that said intermittent washes can be carried out at any temperature with view on attaining high washing efficiency and minimized cost. Further, the person skilled in the art will know that various salts, acids and bases may be added to the washing solutions to increase the efficiency of the washes, as well as that the aforementioned washes can be carried out in a batch mode, flow mode or any combination thereof in order to attain optimal washing efficiency. Furthermore, the person skilled in the art will know that any type of purified, distilled or regular water can be used in the context of aqueous Fmoc/t-Bu SPPS. Furthermore, washing steps can be more or less intensive, varying from a repetition of several batch washes to a simple draining of the resin, depending on the expected purity and of the operating conditions selected for the coupling and deprotection. Washes after coupling can be conveniently omitted and replaced for example by a quenching of residual activated amino acid derivatives and capping agents (Green Chem. 2019, 21, 2594-2600). A one pot coupling/deprotection is possible under certain conditions known by the person skilled in the art (WO2017/070512).

### Regeneration

A further embodiment of the present invention relates to a process for the regeneration of spent solutions emanating from the solid-phase peptide synthesis method described herein. By spent reaction solution is meant any solution separated from the resin in course of the repeating cycles forming the target peptide.

More specifically, the regeneration process comprises a unit operation capable of removing essentially all basic and acid compounds capable of negatively impacting various steps in the Fmoc SPPS disclosed herein. By essentially all is meant at least about 80 wt % of basic and acid compounds capable of negatively impacting various steps in the Fmoc SPPS, more specifically at least about 90 wt %, suitably at least about 95 wt %, typically at least about 99 wt %.

The unit operation may comprise but are not limited to, any one or a combination of distillation, filtration, membrane separation, and ion exchange.

Suitably, the regeneration process comprises ion exchange, that is removal/separation based on charge. Preferably, the ion exchange comprises resins comprising anionic and cationic functional groups. The ionic exchange may comprise at least one resin comprising anionic and cationic functional groups. However, the ion exchange may also comprise at least two resins, one first resin which comprises cationic functional groups without anionic functional groups and one second resin comprising anionic functional groups not having cationic groups.

Managing waste produced in aqueous organic synthesis is challenging (Org. Process Res. Dev. 2021, 25, 900-915). Therefore, developing simple, inexpensive and energy efficient processes for recycling and reusing of waste formed in the context of aqueous SPPS is of utmost importance to enable a truly green, cost-efficient aqueous peptide synthesis methodology. To this end a concept was devised in which the liquid waste produced within the realms of aqueous Fmoc/t-Bu SPPS is recycled by a simple post-synthesis processing methodology, upon which the resulting aqueous solutions are reused in aqueous Fmoc/t-Bu SPPS without any negative effects on the crude peptides produced in the recycled waste as compared to the crude peptide produced in the virgin aqueous solvent (FIG. 2). Specifically, a methodology was developed in which the aqueous SPPS waste is passed through a suitable ion exchange (IEX) stationary phase(s), a processing stage which removes all acidas well as basic compounds capable of disrupting various steps of an aqueous Fmoc/t-Bu SPPS process. More specifically, the aqueous waste is passed through either a cation exchange (CEX) and anion exchange (AEX) resins or through a mixed IEX resin. Subsequently, as the content of the organic cosolvent can be altered upon passing the waste through IEX resin(s), the content of the organic cosolvent is adjusted so that the water/cosolvent ratio is the same as in the virgin water/cosolvent mixture used in aqueous Fmoc/t-Bu SPPS. The SPPS waste stream thus treated is then reused in the synthesis without any further processing or manipulation. The IEX stationary phases are reconditioned by methods customary in IEX aided downstream processing of peptides and proteins and the IEX stationary phases thus reconditioned are then used again to recycle the waste stream from aqueous Fmoc/t-Bu SPPS.

Alternatively, the waste stream produced in aqueous Fmoc/t-Bu SPPS can be recycled by other methods commonly used for recycling of aqueous wastes, for example by using methodologies based on distillation (see e.g. Molecules, 2020, 25, 5264) and membrane separation (see e.g. ACS Macro Lett. 2020, 9, 1709) technologies.

The regeneration process is capable of producing regenerated aqueous solutions which may be successfully used for Fmoc SPPS.

Thus, according to yet a further embodiment, the invention also relates to a regenerated aqueous solution for use in the SPPS method as defined by the instant invention.

### DESCRIPTION OF FIGURES

FIG. 1 Schematic representation of aqueous Fmoc/t-Bu SPPS.

FIG. 2 Schematic representation of recycling and reusing of SPPS waste stream in the context of aqueous Fmoc/t-Bu SPPS.

FIG. 3 Selection of activation/coupling agents: A: COMU (1-cyano-2-ethoxy-2-oxoethylidenaminooxy)dimethylamino-morpholino-carbenium hexafluorophosphate); B: HDMC (6-chloro-1-((dimethylamino)(morpholino)-methylene)-1H-benzotriazolium TFFHhexafluorophosphate 3-oxide; C: DMCH (N-(chloro(morpholino)methylene)-N-methylmethanaminium hexafluorophosphate); D: TCFH (N,N,N′,N′-Tetramethylchloroformamidinium Hexafluorophosphate); E: TFFH (tetramethylfluoroformamidinium hexafluorophosphate)

FIG. 4 HPLC chromatogram of crude Leu-enkephalin amide synthesized in H₂O/PC (4:1). Top: blank (10% AcOH/40% MeCN), bottom: Leu-enkephalin amide.

FIG. 5 UV chromatogram from LC-HRMS analysis of crude Leu-enkephalin amide. Upper trace, blank (10% AcOH/40% MeCN), lower trace, Leu-enkephalin amide.

FIG. 6 MS spectrum [M+H]⁺ of the main peak (Rt 13.87 min) for FIG. 5 crude Leu-enkephalin amide, calcd MS [M+H]⁺ 555.2926, found 555.2923.

FIG. 7 HPLC chromatogram of blank solvent (MeCN).

FIG. 8 HPLC chromatogram of 50 μL virgin PC/H₂O (1:4) in MeCN (1.0 mL). Integrated area of methyl-5-(dimethylamino)-2-methyl-5-oxopentanoate (Rt=10.2 min), the main constituent of PC, was 156.2 mAU×min.

FIG. 9 HPLC chromatogram of 50 μL SPPS waste in MeCN (1.0 mL). Integrated area of methyl-5-(dimethylamino)-2-methyl-5-oxopentanoate (Rt=10.2 min), the main constituent of PC, was 123.6 mAU×min. The content of PC in the SPPS waste was estimated to be around 16% (v/v). The SPPS waste was used as such in the Table 4, entry 2 Fmoc-Gly-OH+TentaGel S NH₂ coupling.

FIG. 10 HPLC chromatogram of 50 μL SPPS waste filtered through SP-Toyopearl-650C IEX resin in MeCN (1.0 mL). Integrated area of methyl-5-(dimethylamino)-2-methyl-5-oxopentanoate (Rt=10.2 min), the main constituent of PC, was 148.0 mAU×min and the content of PC in this recycled SPPS waste was estimated to be around 19% (v/v). The content of PC in this recycled SPPS waste was adjusted to 20% (v/v) before using it in Table 4, entry 3 Fmoc-Gly-OH+TentaGel S NH₂ coupling.

FIG. 11 HPLC chromatogram of 50 μL SPPS waste filtered through QAE-Toyopearl-550C IEX resin in MeCN (1.0 mL). Integrated area of methyl-5-(dimethylamino)-2-methyl-5-oxopentanoate (Rt=10.2 min), the main constituent of PC, was 84.6 mAU×min and the content of PC in this recycled SPPS waste was estimated to be around 11% (v/v). The content of PC in this recycled SPPS waste was adjusted to 20% (v/v) before using it in Table 4, entry 4 Fmoc-Gly-OH+TentaGel S NH₂ coupling.

FIG. 12 HPLC chromatogram of 50 μL SPPS waste filtered through SP-Toyopearl-650C and QAE-Toyopearl-550C IEX resins in MeCN (1.0 mL). Integrated area of methyl-5-(dimethylamino)-2-methyl-5-oxopentanoate (Rt=10.2 min), the main constituent of PC, was 56.7 mAU×min and the content of PC in this recycled SPPS waste was estimated to be around 7% (v/v). The content of PC in this recycled SPPS waste was adjusted to 20% (v/v) before using it in Table 4, entry 5 Fmoc-Gly-OH+TentaGel S NH₂ coupling.

FIG. 13 HPLC chromatogram of 50 μL SPPS waste filtered through Amberlite MB-6113 IEX resin in MeCN (1.0 mL). Integrated area of methyl-5-(dimethylamino)-2-methyl-5-oxopentanoate (Rt=10.2 min), the main constituent of PC, was 106.2 mAU×min and the content of PC in this recycled SPPS waste was estimated to be around 14% (v/v). The content of PC in this recycled SPPS waste was adjusted to 20% (v/v) before using it in Table 4, entry 6 Fmoc-Gly-OH+TentaGel S NH₂ coupling.

FIG. 14 HPLC chromatogram of crude Leu-enk amide synthesized in recycled SPPS waste. Top: blank (10% AcOH/40% MeCN); Bottom: Leu-enk amide.

FIG. 15 Integrated area (48.4 mAu×min) of DBF peak (18.25 min) from determination of Fmoc content on fully loaded reference Fmoc-Gly TentaGel S resin. The conversions of coupling experiments shown in Table 2 were calculated by comparing the Fmoc contents on the Fmoc-Gly TentaGel S resins obtained to the Fmoc content on the reference Fmoc-Gly TentaGel S resin

FIG. 16 HPLC chromatogram of crude Leu-enkephalin amide synthesized in H₂O/NBP (4:1) on TG S NH₂ resin. Top blank (10% AcOH/40% MeCN), bottom, Leu-enkephalin amide.

FIG. 17 HPLC chromatogram of crude Leu-enkephalin amide synthesized in H₂O/MeCN (4:1) on TG S NH₂ resin. Top, blank (10% AcOH/40% MeCN), bottom, Leu-enkephalin amide.

FIG. 18 HPLC chromatogram of crude Leu-enkephalin amide synthesized in H₂O/DMPU (4:1) on TG S NH₂ resin. Top, blank (10% AcOH/40% MeCN), bottom, Leu-enkephalin amide.

FIG. 19 HPLC chromatogram of crude Leu-enkephalin amide synthesized in H₂O/DMSO (4:1) on TG S NH₂ resin. Top, blank (10% AcOH/40% MeCN), bottom, Leu-enkephalin amide.

FIG. 20 UV chromatogram from LC-MS analysis of crude Leu-enkephalin amide synthesized in H₂O/NBP (4:1) on TG S NH₂ resin. Upper diagram, blank (10% AcOH/40% MeCN), lower diagram, Leu-enkephalin amide.

FIG. 21 MS spectrum [M+H]⁺ of the main peak (Rt 9.74 min) for FIG. 20 crude Leu-enkephalin amide, calcd MS [M+H]⁺ 555.2926, found 555.2.

FIG. 22 UV chromatogram from LC-MS analysis of crude Leu-enkephalin amide synthesized in H₂O/MeCN (4:1) on TG S NH₂ resin. For blank (10% AcOH/40% MeCN) see FIG. 20.

FIG. 23 MS spectrum [M+H]+ of the main peak (Rt 9.75 min) for FIG. 22 crude Leu-enkephalin amide, calcd MS [M+H]+ 555.2926, found 555.2.

FIG. 24 UV chromatogram from LC-MS analysis of crude Leu-enkephalin amide synthesized in H₂O/DMPU (4:1) on TG S NH₂ resin. For blank (10% AcOH/40% MeCN) see FIG. 20.

FIG. 25 MS spectrum [M+H]+ of the main peak (Rt 9.74 min) for FIG. 24 crude Leu-enkephalin amide, calcd MS [M+H]+ 555.2926, found 555.2.

FIG. 26 UV chromatogram from LC-MS analysis of crude Leu-enkephalin amide synthesized in H₂O/DMSO (4:1) on TG S NH₂ resin. For blank (10% AcOH/40% MeCN) see FIG. 20.

FIG. 27 MS spectrum [M+H]+ of the main peak (Rt 9.76 min) for FIG. 20 crude Leu-enkephalin amide, calcd MS [M+H]+ 555.2926, found 555.1.

FIG. 28 UV chromatogram from LC-HRMS analysis of crude Leu-enkephalin amide synthesized in H₂O/PC (4:1) on TG S NH₂ resin. Upper trace, blank (10% AcOH/40% MeCN), lower trace, Leu-enkephalin amide.

FIG. 29 MS spectrum [M+H]⁺ of the main peak (Rt 13.87 min) for FIG. 28 crude Leu-enkephalin amide, calcd MS [M+H]⁺ 555.2926, found 555.2923.

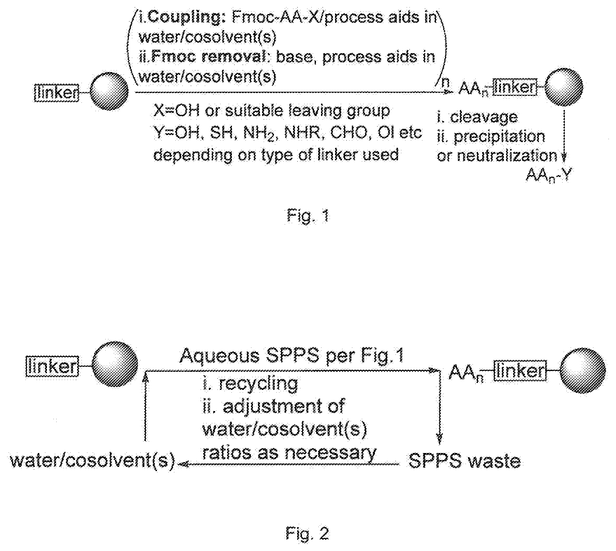

### EXPERIMENTAL SECTION

### 1. General Information

All reagents, reactants and solvents were from standard suppliers of raw materials for peptide synthesis and were used as such. PolarClean™ was from Solvay. Additional co-solvents used were NBP, MeCN, DMPU and DMSO. All coupling reagents were from Luxembourg Bio Technologies. Tap water was used throughout. The SPPS resins tested were from following suppliers: Agilent (0.44 mmol/g AM PS/DVB resin and 0.76 mmol/AmphiSpheres NH₂ resin), Rapp Polymere GmbH (0.27 mmol/g TentaGel S NH₂ resin), Sigma Aldrich (1.00 mmol/g JandaJel NH₂ resin), Hecheng (0.55 mmol/g DEG AM resin), Aapptec (0.34 mmol/g NH₂ OctaGel resin) and PCAS BioMatrix (1.30 mmol/g ChemMatrix NH₂ resin).

The IEX resins examined in SPPS waste recycling experiments were from Tosoh Bioscience (SP-Toyopearl-650C and QAE-Toyopearl-550C) and Supelco (Amberlite MB-6113).

All coupling and Fmoc removal experiments as well as Leu-enkephalin syntheses were carried out in sealed fritted syringes on a temperature controlled PLS4x6 Activo-PLS parallel reaction synthesizer (Activotec). For all experiments the agitation of the reactions was carried out by shaking at 350 rpm.

All HPLC analyses were carried out on a Waters Alliance instrument using Waters XSelect CSH130 C18 2.5μ 4.6×150 mm column, TFA/H2O (0.1:100, A), TFA/MeCN (0.08:100 B) as buffers, 100% B over 15 min gradient, flow of 0.5 mL/min, detection at 220 nm and column temperature 30° C.

LC-MS analyses were performed on a Horizon high performance liquid chromatography system (Thermo, Waltham, Massachusetts, U.S) with variable wavelength detector connected to a Q-Exactive orbitrap mass spectrometry system (Thermo, Waltham, Massachusetts, U.S). The mass spectrometry system was operated in a positive mode using sheath ESI, mass accuracy 5 ppm, resolution up to 140 000 ppm. The following source settings were used: sheath gas flow rate 35, aux gas flow rate 10, sweep gas flow rate 1, spray voltage (kV) 3.50, capillary temp. 250° C., S-lens RF level 50.0 and Aux gas heater temp. 200° C.

Experimental conditions: column: Waters CSH 2.5 um 4.6×150 mm; column temperature: 20° C.; injection volume: 1 μL; sampler temperature: 10° C.; MS mode: positive 50-3200; DAD: 220 nm; data rate: 10 Hz; detector cell: standard cell 1 uL; flow: 0.2 ml/min; jet weaver: V150 mixer; mobile phase A: 0.3% TFA in 90% water/MeCN, mobile phase B: 0.30% TFA in 10% water/MeCN. Gradient (Time(min), % B): 0, 0; 1, 0; 42, 100; 47, 100; 47.1, 0; 58, 0.

LC-MS analyses were performed on a Thermoscientific MSQ Plus in a positive mode (ESI) coupled with Dionex UltiMate 3000 using the following experimental conditions: Waters XSelect Peptide CSH130 C18 XP 2.5μ 4.6×150 mm column, TFA/H2O (0.1:100, A), TFA/MeCN (0.1:100 B) as buffers, 5% B to 90% B over 10 min gradient, flow of 2.0 mL/min, detection at 220 nm and column temperature 30° C.

### 2. Swelling of SPPS Resins in Different Solvents

In a 10 mL fritted syringe at rt (room temperature), 1.0 g of each resin was swollen in a suitable amount of the stated solvent. For the determination of swelling of the ChemMatrix resin 0.5 g was used due to the high swelling properties of this polymer support. The syringe was sealed and shaken at rt for one hour after which the syringe with the swollen resin was allowed to stand at rt for one hour and the volume occupied by the swollen resin was determined.

### List of Tested Resins

AM PS/DVB: Aminomethyl (AM) PS/divinylbenzene (DVB)
JandaJel NH₂: diethylene glycol (DEG) containing PS resin
DEG AM: diethylene glycol (DEG) containing PS resin
TG S NH₂: TentaGel S NH₂ resin
AmphiSpheres NH₂
NH₂-OctaGel
ChemMatrix NH₂

### List of Tested Co-Solvents

PolarClean™ Solvay
NBP
MeCN
DMPU
DMSO

TABLE 1
Swelling (mL g⁻¹) of SPPS resins.

| | | Co- | Co-solvent/H₂O ratio | | |
|---|---|---|---|---|---|
| Entry | Resin | solvent | 1:0 | 1:1 | 0:1 |
| 1 | AM PS/DVB | NBP | 8.0 | 1.8 | 1.8 |
| 2 | AM PS/DVB | PC | 8.0 | 3.1 | 1.8 |
| 3 | JandaJel NH₂ | PC | 9.0 | 2.0 | 2.0 |
| 4 | DEG AM | PC | 5.5 | 1.7 | 1.7 |
| 5 | TG S NH₂ | PC | 1.9 | 4.9 | 3.3 |
| 6 | TG S NH₂ | NBP | 2.2 | 5.1 | 3.3 |
| 7 | TG S NH₂ | MeCN | 4.4 | 5.0 | 3.3 |
| 8 | AmphiSpheres NH₂ | PC | 6.5 | 2.0 | 2.0 |
| 9 | NH2-OctaGel | PC | 6.5 | 2.5 | 2.0 |
| 10 | ChemMatrix NH₂ | PC | 3.6 | 15.0 | 15.8 |

### 3. Model Coupling of Fmoc-Gly-OH on TentaGel S NH₂ Resin

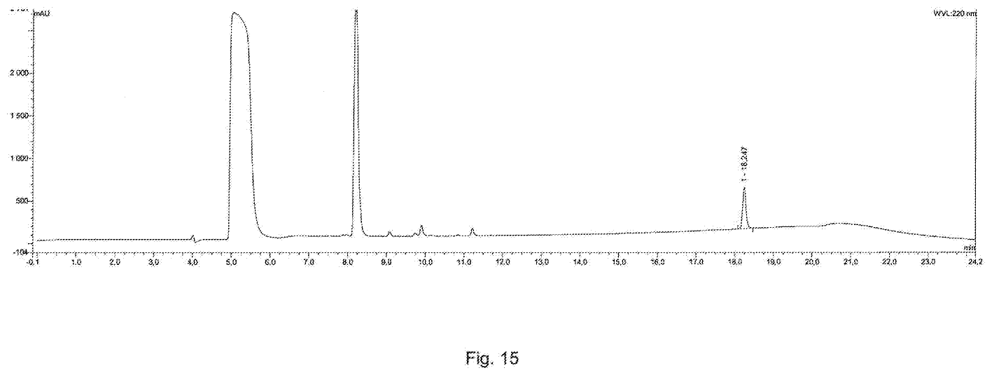

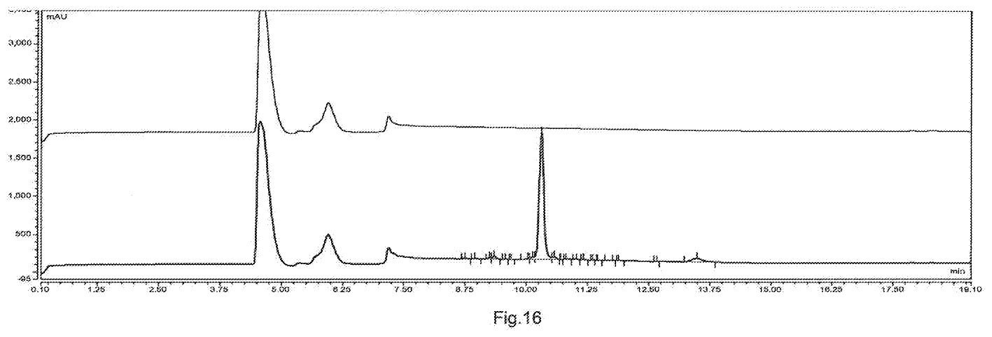

General protocol: 1.0 g of 0.27 mmol/g TentaGel S NH₂ resin (0.27 mmol) was weighed into a fritted syringe. A reaction solvent (8 mL) as specified in Table 2 was added to the resin and the resulting slurry was shaken at rt for 1 h and drained. Next, Fmoc-Gly-OH, coupling agent and base were added in the amounts specified in Table 2 after which a solvent (5 mL) specified in Table 2 was added. The syringe was sealed and the resulting slurry was agitated by shaking at the temperature stated in Table 2 for 1 h. For the Table 2, entry 5 experiment the stated amounts of COMU and collidine respectively were added during the coupling experiments at the stated intervals. Upon completion of a coupling experiment the resin was drained, washed with the reaction solvent (3×10 mL), NBP (5×10 mL) and i-PrOH (5×10 mL) and dried in vacuo to constant weight. The coupling conversions given in Table 2 were calculated using a fully coupled Fmoc-Gly TentaGel S resin as a reference using a method for determination of Fmoc content on peptide resins in which the dibenzofulvene (DBF) liberated from the Fmoc containing resins by the action of the strong base 1,8-diazabicyclo[5.4.0]undec-7-ene (DBU) is quantified employing a previously employed protocol. Thus, a sample of the fully coupled Fmoc-Gly TentaGel S resin was obtained by coupling of 1.0 g of 0.27 mmol/g TentaGel S NH₂ resin (0.27 mmol) with 4 equiv Fmoc-Gly-OH and 4 equiv DIC/Oxyma (1:1) in 5 mL DMF for 1 h at rt followed by washing with DMF (5×10 mL) and i-PrOH (5×10 mL) and drying in vacuo to constant weight. The Fmoc content on this reference Fmoc-Gly TentaGel S resin was determined by weighing out 50.0 mg of the resin, stirring with 2% (v/v) DBU/DMF (2 mL) for 30 min and diluting the reaction mixture to 10.0 mL with MeCN. The solution thus obtained was analyzed by the HPLC method. The peak for the dibenzofulvene (DBF) was integrated (FIG. 15) and used as a reference standard (100% conversion) for the DBF peaks of the samples of Fmoc-Gly TentaGel S resins obtained in the coupling experiments depicted in Table 2. The completion of the coupling experiments shown in Table 2 was also assessed using a qualitative (ninhydrin) color test.

TABLE 2
Assessment of coupling of Fmoc-Gly-OH with TG S NH2 resin. ^{[a]}

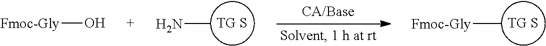

| | | | | Equiv | | |
|---|---|---|---|---|---|---|
| | | | | Fmoc- | Fmoc-Gly- | Conv. |
| Entry | Solvent | CA | Base | Gly-OH | OH/CA/Base | (%)^{[b]} |
| 1 | H₂O | COMU | collidine | 1.1 | 1:1:3 | 37.1 |
| 2 | H₂O/PC (4:1) | COMU | collidine | 1.1 | 1:1:3 | 53.1 |
| 3 | H₂O/PC (1:4) | COMU | collidine | 1.1 | 1:1:3 | 56.4 |
| 4 | PC | COMU | collidine | 1.1 | 1:1:3 | 14.1 |
| 5 | H₂O/PC (4:1) | COMU^{[d]} | collidine | 1.1 | 1:1:3 | 65.1 |
| 6 | H₂O/PC (4:1) | COMU^{[e]} | collidine | 1.1 | 1:1:3 | 57.3 |
| 7 | H₂O/PC (4:1) | COMU | collidine | 4.4 | 1:1:3 | >99^{[c]} |
| 8 | H₂O/PC (4:1) | HDMC | collidine | 1.1 | 1:1:3 | 26.3 |
| 9 | H₂O/PC (4:1) | DMCH | collidine | 1.1 | 1:1:3 | 85.0 |
| 10 | H₂O/PC (4:1) | TCFH | collidine | 1.1 | 1:1:3 | 83.3 |
| 11 | H₂O/PC (4:1) | TFFH | collidine | 1.1 | 1:1:3 | 41.5 |
| 12 | H₂O/PC (4:1) | TCFH | none | 1.5 | 1:1:0 | 8.1 |
| 13 | H₂O/PC (4:1) | TCFH | collidine | 1.5 | 1:1:1.5 | 50.0 |
| 14 | H₂O/PC (4:1) | TCFH | collidine | 1.5 | 1:1:3 | >99^{[c]} |
| 15 | H₂O/PC (4:1) | TCFH | collidine | 1.5 | 1:1:4.5 | >99^{[c]} |
| 16 | H₂O/PC (4:1) | TCFH | DIEA | 1.5 | 1:1:3 | 29.5 |
| 17 | H₂O/PC (4:1) | TCFH | NMM | 1.5 | 1:1:3 | 32.0 |
| 18 | H₂O/PC (4:1) | TCFH | NMI | 1.5 | 1:1:3 | 40.4 |
| 19 | H₂O/PC (4:1) | TCFH | collidine | 1.3 | 1:1:3 | >99^{[c]} |
| 20 | H₂O/PC (4:1) | TCFH | collidine | 1.2 | 1:1:3 | 90.6 |

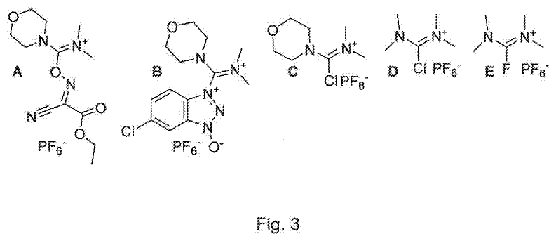

^{[a]} For names and structures of all CAs see Fig. 3;
^{[b]}Determined by quantifying Fmoc content on Fmoc-Gly TG S resins;
^{[c]}Complete according to a negative (ninhydrin) color test;
^{[d]}COMU and collidine were added in 4 equal portions at t = 0, 15, 30 and 45 min;
^{[e]}Carried out at 40° C.

### 4. Fmoc Deprotection of Fmoc-RMG TentaGel S Resin (RMG: Ramage Linker)

Fmoc-RMG TentaGel S resin was prepared from TentaGel S NH₂ resin and Ramage linker (Fmoc-RMG-OH) according to a previously reported protocol employing DIC and Oxyma as coupling agents (Green Chem. 2019, 21, 2594). Next, the Fmoc deprotection experiments described in Table 3 were carried out as follows: 1.0 g of 0.18 mmol/g Fmoc-RMG TentaGel S resin (0.18 mmol) was weighed into a fritted syringe. A reaction solvent (8 mL) as specified in Table 3 was added to the resin and the resulting slurry was shaken at rt for 1 h and drained. 10 mL of 5% (v/v) 4-methylpiperidine (4-MP) in solvent as specified in Table 3 was added, the syringe was sealed and the resulting slurry was agitated by shaking at the temperature stated in Table 3 for 15 min. For the experiment described in Table 3, entry 5 the Fmoc removal treatment was repeated. Upon completion of an Fmoc removal experiment the resin was drained, washed with the reaction solvent (3×10 mL), NBP (5×10 mL) and i-PrOH (5×10 mL) and dried in vacuo to constant weight. The Fmoc removal conversions stated in Table 3 were calculated using the starting Fmoc-RMG TentaGel S resin as a reference using the method for determination of Fmoc content on peptide resins. Thus, the Fmoc content on the reference Fmoc-RMG TentaGel S resin was determined by weighing out 50 mg of the resin, stirring it with 2% (v/v) DBU/DMF (2 mL) for 30 min and diluting the reaction mixture to 10.0 mL with MeCN. The solution thus obtained was analyzed by the HPLC method. The peak for the dibenzofulvene (DBF) was integrated and used as a reference standard for the samples of H-RMG TentaGel S resin products obtained in the Fmoc removal experiments depicted in Table 3. The Fmoc removal conversions (%) were calculated as 100×(DBF area for the H-RMG resin product/DBF area for the Fmoc-RMG resin starting material).

TABLE 3
Fmoc removal from Fmoc RMG TG S resin.

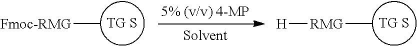

| Entry | Solvent | Time (min) | Temperature | Conv. (%)^{[a]} |
|---|---|---|---|---|
| 1 | H₂O | 15 | rt | 71.5 |
| 2 | H₂O/PC (4:1) | 15 | rt | 94.7 |
| 3 | H₂O/PC (1:4) | 15 | rt | 90.9 |
| 4 | PC | 15 | rt | 88.0 |
| 5 | H₂O/PC (4:1) | 2 × 15 | rt | 99.8 |
| 6 | H₂O/PC (4:1) | 15 | 40° C. | 99.2 |

^{[a]}Determined by quantifying Fmoc content on H-RMG TG S resins.

### 5. SPPS of Leu-Enkephalin Amide in H₂O/Co-Solvent (4:1)

The classical Leu enkephalin amide was used as substrate employing RMG TG S as starting resin, H₂O/co-solvent (4:1) as solvent and 1.3 equiv of Fmoc-AA-OH/TCFH/collidine (1:1:3) for couplings and 4-MP (5% v/v) for Fmoc removals (Scheme 1).

Co-solvents were selected from NBP, PC, MeCN, DMPU and DMSO.

1.0 g of 0.18M Fmoc-RMG TentaGel S resin (0.18 mmol) was weighed into a fritted syringe and 10 mL H₂O/co-solvent (4:1) was added. The syringe was sealed and the resulting slurry was shaken at rt for 1 h and drained. Next, four amino acid (AA) coupling cycles were carried out as outlined in Scheme 1 as follows:
i) Fmoc deprotection with 10 mL 5% 4-MP (v/v) in co-solvent/H₂O (1:4) for 2×15 min at 40° C.;
ii) 4×10 mL H₂O/co-solvent (4:1) washes, 5 min each. The first three washes were carried out at 40° C. and the fourth wash was carried out at rt;
iii) AA coupling carried out by weighing in 1.3 equiv Fmoc-AA-OH (0.23 mmol) and TCFH (0.23 mmol, 65.6 mg) followed by adding 3.9 equiv collidine (0.70 mmol, 92.8 μL) and 5 mL H₂O/co-solvent (4:1). The syringe was sealed and the resulting slurry was stirred at rt for 1 h and drained. The amounts of AAs used were as follows: 1st AA cycle, Fmoc-Leu-OH, 81.3 mg; 2nd AA cycle, Fmoc-Phe-OH, 90.7 mg; 3rd AA cycle, Fmoc-Gly-Gly-OH, 85.1 mg; 4th AA cycle, Fmoc-Tyr (t-Bu)-OH, 107.5 mg;
iv) 1×10 mL co-solvent/H₂O (1:4) wash, 5 min at rt.

Next, an Fmoc deprotection and 4×10 mL H₂O/co-solvent (4:1) washes were carried out as described above in i) & ii) followed by 4×10 mL i-PrOH washes. The final resin was dried to constant weight in vacuo.

The synthesis carried out in H₂O/NBP (4:1) (Table 4, entry 1) afforded 0.96 g of Leu-enkephalin RMG TentaGel S peptide resin. During the synthesis, the progress of all couplings and Fmoc deprotections was monitored by qualitative colorimetric tests (ninhydrin), thereby slightly decreasing the total amount of the peptide resin obtained. 100 mg of the Leu-enkephalin amide resin was weighed into a fritted syringe, 1.0 mL of TFA/TIS/H₂O (90:5:5) was added and the resulting slurry was shaken at rt for 2 h. The spent resin was filtered off and washed with 2×0.5 mL TFA. The combined volatiles were removed in vacuo and the crude peptide was precipitated by 2×20 mL diethyl ether (Et₂O) affording 10.9 mg (88%) of crude Leu-enkephalin amide.

The synthesis carried out in H₂O/PC (4:1) (Table 4, entry 2) afforded 1.06 g of Leu-enkephalin RMG TentaGel S peptide resin. 100 mg of the Leu-enkephalin amide resin was weighed into a fritted syringe, 1.0 mL of TFA/TIS/H₂O (90:5:5) was added and the resulting slurry was shaken at rt for 2 h. The spent resin was filtered off and washed with 2×0.5 mL TFA. The combined volatiles were removed in vacuo and the crude peptide was precipitated by 2×20 mL diethyl ether (Et₂O) affording 9.7 mg (85%) of crude Leu-enkephalin amide.

The synthesis carried out in H₂O/MeCN (4:1) (Table 4, entry 3) afforded 0.98 g of Leu-enkephalin RMG TentaGel S peptide resin. During the synthesis, the progress of all couplings and Fmoc deprotections was monitored by qualitative colorimetric tests (ninhydrin), thereby slightly decreasing the total amount of the peptide resin obtained. 100 mg of the Leu-enkephalin amide resin was weighed into a fritted syringe, 1.0 mL of TFA/TIS/H₂O (90:5:5) was added and the resulting slurry was shaken at rt for 2 h. The spent resin was filtered off and washed with 2×0.5 mL TFA. The combined volatiles were removed in vacuo and the crude peptide was precipitated by 2×20 mL diethyl ether (Et₂O) affording 10.8 mg (88%) of crude Leu-enkephalin amide.

The synthesis carried out in H₂O/DMPU (4:1) (Table 4, entry 4) afforded 0.97 g of Leu-enkephalin RMG TentaGel S peptide resin. During the synthesis the progress of all couplings and Fmoc deprotections was monitored by qualitative colorimetric tests (ninhydrin), thereby slightly decreasing the total amount of the peptide resin obtained. 100 mg of the Leu-enkephalin amide resin was weighed into a fritted syringe, 1.0 mL of TFA/TIS/H₂O (90:5:5) was added and the resulting slurry was shaken at rt for 2 h. The spent resin was filtered off and washed with 2×0.5 mL TFA. The combined volatiles were removed in vacuo and the crude peptide was precipitated by 2×20 mL diethyl ether (Et₂O) affording 8.7 mg (70%) of crude Leu-enkephalin amide.

The synthesis carried out in H₂O/DMSO (4:1) (Table 4, entry 5) afforded 0.96 g of Leu-enkephalin RMG TentaGel S peptide resin. During the synthesis, the progress of all couplings and Fmoc deprotections was monitored by qualitative colorimetric tests (ninhydrin), thereby slightly decreasing the total amount of the peptide resin obtained. 100 mg of the Leu-enkephalin amide resin was weighed into a fritted syringe, 1.0 mL of TFA/TIS/H₂O (90:5:5) was added and the resulting slurry was shaken at rt for 2 h. The spent resin was filtered off and washed with 2×0.5 mL TFA. The combined volatiles were removed in vacuo and the crude peptide was precipitated by 2×20 mL diethyl ether (Et₂O) affording 11.1 mg (88%) of crude Leu-enkephalin amide

TABLE 4
Overview of SPPS of Leu enk NH₂ in H₂O/co-solvent
(4:1) on TentaGel S NH₂ resin.

| | | | | LC-MS |
|---|---|---|---|---|
| | | TG S NH₂ swelling | Leu enk NH₂ | mass of |
| | | (mL g⁻¹) in H₂O/ | HPLC purity | the main |
| Entry | Co-solvent | co-solvent (4:1) | (%) | peak^{a} |
| 1 | NBP | 5.0 | 86 | 555.2^{b} |
| 2 | PC | 4.5 | 86 | 555.2923^{c} |
| 3 | MeCN | 4.5 | 86 | 555.2^{b} |
| 4 | DMPU | 4.0 | 86 | 555.2^{b} |
| 5 | DMSO | 3.7 | 44 | 555.1^{b} |

^{a}calcd MS [M + H]⁺ 555.2926;
^{b}determined by LC-MS;
^{c}determined by LC-HRMS.

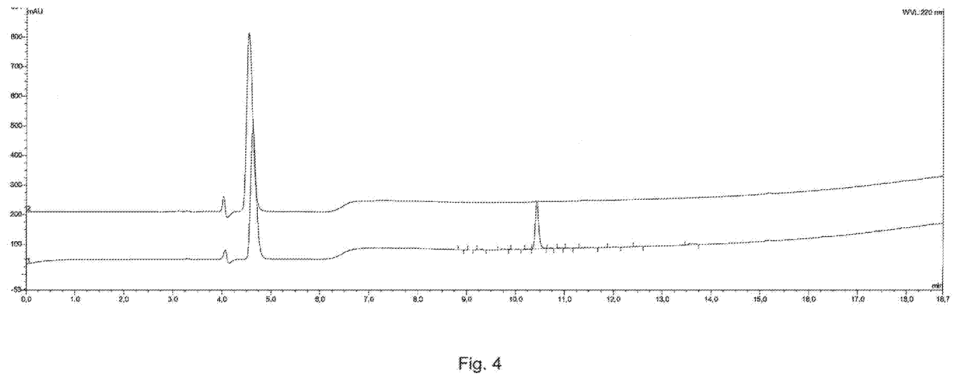

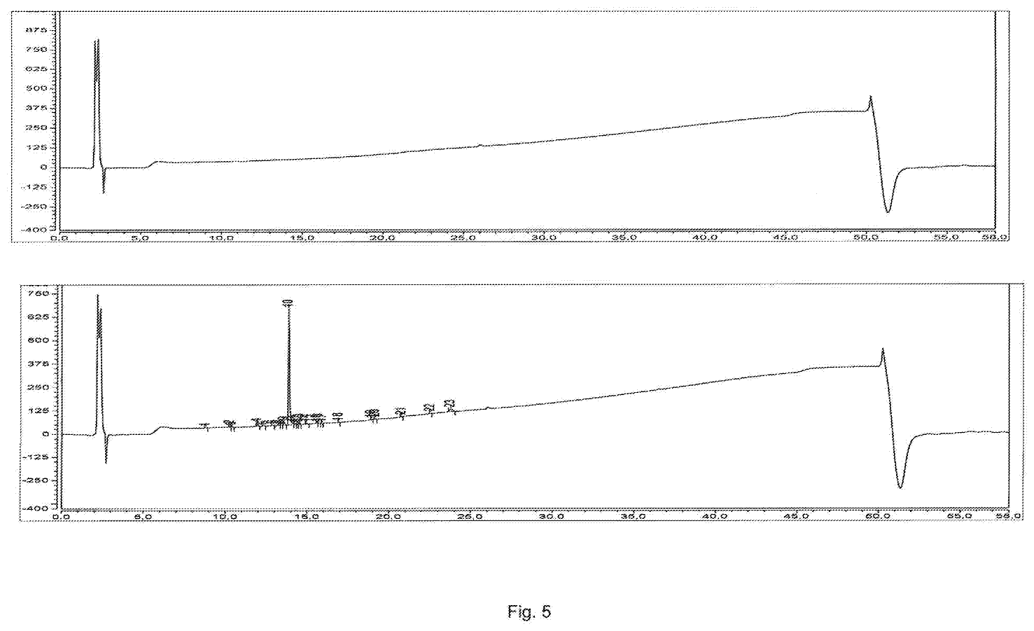

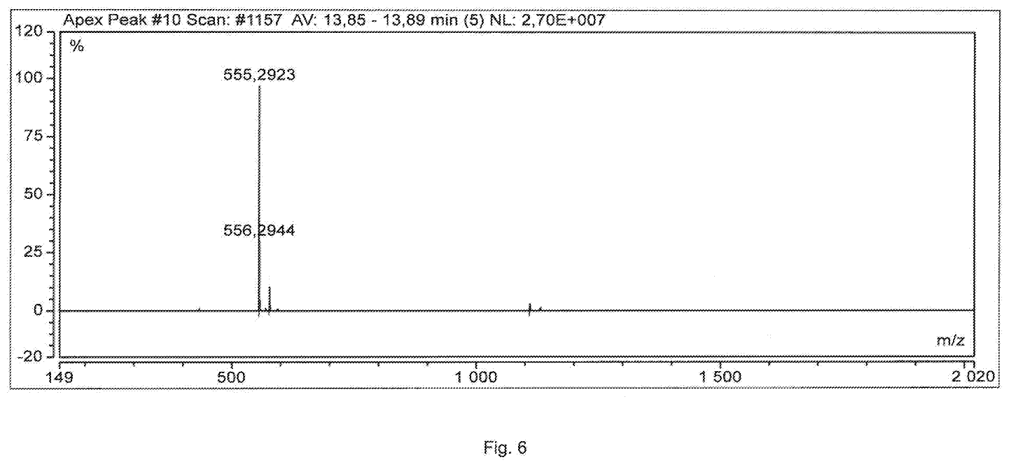

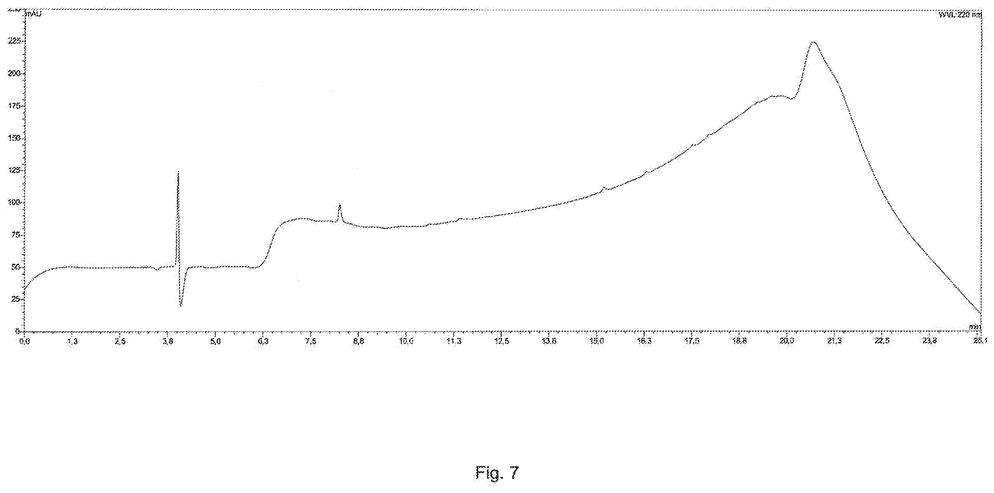

The total amount of generated H₂O/PC containing SPPS waste was about 370 mL. The washings from the i-PrOH washes of the final peptide resin were not pooled with the H₂O/PC containing SPPS waste. 100 mg of the Leu-enkephalin amide resin was weighed into a fritted syringe, 1.0 mL of TFA/TIS/H₂O (90:5:5) was added and the resulting slurry was shaken at rt for 2 h. The spent resin was filtered off and washed with 2×0.5 mL TFA. The combined volatiles were removed in vacuo and the crude peptide was precipitated by 2×20 mL diethyl ether (Et₂O) affording 9.7 mg (85%) of crude Leu-enkephalin amide. The HPLC purity of the crude product was 86% (FIG. 4 and Table 6) and the identity of the target peptide was confirmed by LC-HRMS (FIGS. 5 and 6 and Table 15).

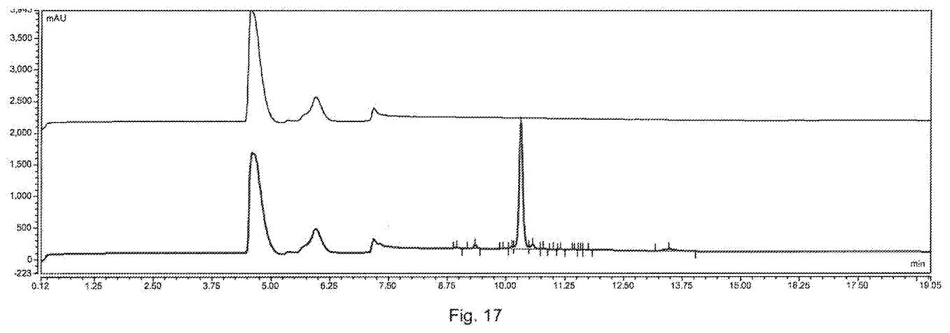

TABLE 5
Integrated peaks for FIG. 16 crude Leu-enkephaline amide (co-solvent: NBP).

| | Ret. Time | | Rel. Area | Area | Height | | Width | Asym. | Resol. | Plates |
|---|---|---|---|---|---|---|---|---|---|---|
| No. | min | Amount | % | mAU*min | mAU | Type | (50% min) | EP | EP | EP |
| 1 | 8.751 | n.a. | 0.41 | 0.8893 | 11.46 | BMB* | 0.073 | 1.17 | 1.55 | 78930 |
| 2 | 8.954 | n.a. | 0.50 | 1.0756 | 12.52 | BMB* | 0.820 | 1.29 | n.a. | 66822 |
| 3 | 9.246 | n.a. | 0.80 | 1.7221 | 24.23 | BM* | n.a. | n.a. | n.a. | n.a. |
| 4 | 9.343 | n.a. | 1.54 | 3.2978 | 40.04 | MB* | 0.078 | n.a. | 1.86 | 80033 |
| 5 | 9.566 | n.a. | 0.06 | 0.1392 | 2.14 | BMB* | 0.064 | 1.15 | 1.17 | 122803 |
| 6 | 9.687 | n.a. | 0.02 | 0.0495 | 0.82 | BMB* | 0.058 | 1.50 | n.a. | 153196 |
| 7 | 10.037 | n.a. | 0.55 | 1.1824 | 14.68 | BM* | n.a. | n.a. | n.a. | n.a. |
| 8 | 10.134 | n.a. | 1.24 | 2.6542 | 34.79 | M* | n.a. | n.a. | n.a. | n.a. |
| 9 | 10.313 | n.a. | 86.33 | 185.3885 | 1665.83 | M* | 0.103 | 1.04 | n.a. | 55959 |
| 10 | 10.575 | n.a. | 1.49 | 3.2019 | 37.18 | MB* | n.a. | n.a. | n.a. | n.a. |
| 11 | 10.706 | n.a. | 0.03 | 0.0592 | 1.55 | BMB* | 0.038 | 1.50 | 0.73 | 445384 |
| 12 | 10.808 | n.a. | 0.34 | 0.7378 | 6.93 | BMB* | 0.128 | 1.85 | 1.14 | 39779 |
| 13 | 11.027 | n.a. | 0.36 | 0.7690 | 8.00 | BMB* | 0.099 | 0.99 | 0.99 | 68105 |
| 14 | 11.163 | n.a. | 0.31 | 0.6577 | 10.09 | BMB* | 0.062 | 1.37 | 2.03 | 178188 |
| 15 | 11.357 | n.a. | 0.06 | 0.1258 | 2.52 | BMB* | 0.051 | 1.26 | 1.01 | 276781 |
| 16 | 11.459 | n.a. | 0.03 | 0.0628 | 0.78 | BMB* | 0.069 | 2.67 | 2.21 | 154576 |
| 17 | 11.760 | n.a. | 0.13 | 0.2811 | 3.07 | BMB* | 0.092 | 0.79 | 0.71 | 90029 |
| 18 | 11.881 | n.a. | 0.08 | 0.1702 | 1.66 | BMB* | 0.108 | 2.32 | 5.52 | 66799 |
| 19 | 12.672 | n.a. | 0.06 | 0.1320 | 2.17 | BMB* | 0.061 | 0.90 | 3.30 | 240633 |
| 20 | 13.497 | n.a. | 5.66 | 12.1557 | 47.58 | BMB* | 0.234 | 1.18 | n.a. | 18422 |
| Sum | | 0.0000 | 100.00 | 214.7519 | 1928.03 | | | | | |

TABLE 6
Integrated peaks for FIG. 4 crude Leu-enkephalin amide (co-solvent: PC).

| | | Ret. Time | Area | | | Height | Rel. Area | |
|---|---|---|---|---|---|---|---|---|
| No. | Peakname | min | mAU*min | Amount | Type | mAU | % | Resolution |
| 1 | n.a. | 8.881 | 0.0328 | n.a. | BMB* | 0.570 | 0.22 | 2.04 |
| 2 | n.a. | 9.081 | 0.0153 | n.a. | BMB* | 0.263 | 0.10 | 2.07 |
| 3 | n.a. | 9.301 | 0.1583 | n.a. | BMB* | 2.293 | 1.07 | 3.69 |
| 4 | n.a. | 9.739 | 0.2928 | n.a. | BMB* | 3.688 | 1.99 | 2.23 |
| 5 | n.a. | 10.024 | 0.0660 | n.a. | BMB* | 0.757 | 0.45 | 2.02 |
| 6 | n.a. | 10.257 | 0.0566 | n.a. | BMB* | 0.930 | 0.38 | 1.58 |
| 7 | n.a. | 10.435 | 12.7271 | n.a. | BMB* | 158.313 | 86.43 | 2.31 |
| 8 | n.a. | 10.715 | 0.1501 | n.a. | BMB* | 2.123 | 1.02 | 1.64 |
| 9 | n.a. | 10.897 | 0.1027 | n.a. | BMB* | 1.693 | 0.70 | 1.35 |
| 10 | n.a. | 11.065 | 0.0413 | n.a. | BMB* | 0.471 | 0.28 | 1.61 |
| 11 | n.a. | 11.419 | 0.3194 | n.a. | BMB* | 1.935 | 2.17 | 2.39 |
| 12 | n.a. | 12.012 | 0.1509 | n.a. | BMB* | 1.149 | 1.03 | 2.72 |
| 13 | n.a. | 12.483 | 0.0401 | n.a. | BMB* | 0.445 | 0.27 | 6.02 |
| 14 | n.a. | 13.594 | 0.5716 | n.a. | BMB* | 4.156 | 3.88 | n.a. |
| Total: | | | 14.7247 | 0.0000 | | 178.785 | 100.00 | |

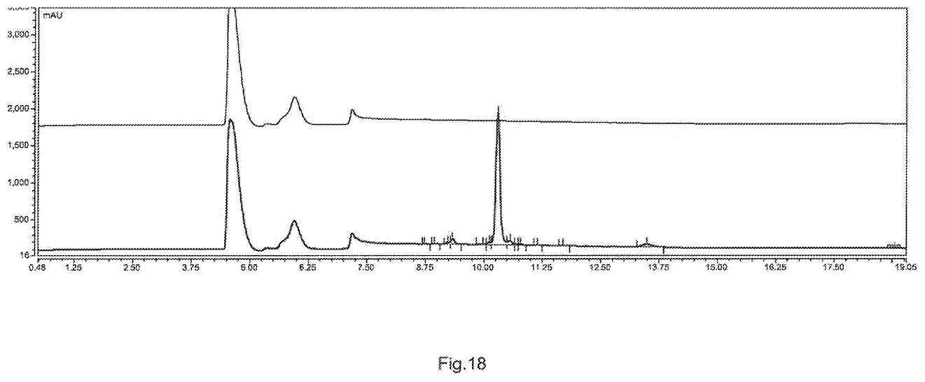

TABLE 7
Integrated peaks for FIG. 17 crude Leu-enkephaline amide (co-solvent MeCN).

| | Ret. Time | | Rel. Area | Area | Height | | Width | Asym. | Resol. | Plates |
|---|---|---|---|---|---|---|---|---|---|---|
| No. | min | Amount | % | mAU*min | mAU | Type | (50% min) | EP | EP | EP |
| 1 | 8.945 | n.a. | 0.79 | 1.7067 | 21.19 | BMB* | 0.075 | 1.40 | 3.15 | 78703 |
| 2 | 9.338 | n.a. | 2.26 | 4.8800 | 57.31 | BMB* | 0.072 | 0.86 | n.a. | 92725 |
| 3 | 9.935 | n.a. | 0.48 | 1.0420 | 8.38 | BM* | n.a. | n.a. | n.a. | n.a. |
| 4 | 10.124 | n.a. | 1.09 | 2.3529 | 33.42 | M* | n.a. | n.a. | n.a. | n.a. |
| 5 | 10.313 | n.a. | 86.14 | 186.0708 | 1995.65 | M* | 0.082 | 1.12 | 1.66 | 86742 |
| 6 | 10.571 | n.a. | 3.49 | 7.5489 | 70.68 | M* | 0.101 | n.a. | 1.51 | 61111 |
| 7 | 10.794 | n.a. | 0.67 | 1.4415 | 18.59 | MB* | 0.073 | n.a. | 1.60 | 120217 |
| 8 | 11.007 | n.a. | 0.06 | 0.1383 | 1.62 | BMB* | 0.084 | 0.93 | 1.20 | 95483 |
| 9 | 11.158 | n.a. | 0.22 | 0.4651 | 6.83 | BMB* | 0.064 | 1.27 | 2.87 | 167143 |
| 10 | 11.459 | n.a. | 0.04 | 0.0773 | 1.31 | BMB* | 0.059 | 1.07 | 1.42 | 206063 |
| 11 | 11.590 | n.a. | 0.02 | 0.0370 | 0.76 | BMB* | 0.049 | 0.98 | 1.16 | 307734 |
| 12 | 11.755 | n.a. | 0.17 | 0.3580 | 3.20 | BMB* | 0.119 | 0.87 | 5.26 | 54061 |
| 13 | 13.463 | n.a. | 4.58 | 9.8908 | 32.20 | BMB* | 0.264 | 1.61 | n.a. | 14397 |
| Sum | | 0.0000 | 100.00 | 216.0093 | 2251.14 | | | | | |

TABLE 8
Integrated peaks for FIG. 18 crude Leu-enkephaline amide (co-solvent DMPU).

| | Ret. Time | | Rel. Area | Area | Height | | Width | Asym. | Resol. | Plates |
|---|---|---|---|---|---|---|---|---|---|---|
| No. | min | Amount | % | mAU*min | mAU | Type | (50% min) | EP | EP | EP |
| 1 | 8.736 | n.a. | 0.10 | 0.2113 | 2.95 | BMB* | 0.069 | 1.34 | 1.55 | 89334 |
| 2 | 8.945 | n.a. | 0.25 | 0.5331 | 5.99 | BMB* | 0.090 | 1.36 | n.a. | 54493 |
| 3 | 9.236 | n.a. | 1.00 | 2.1654 | 29.83 | BM* | n.a. | n.a. | n.a. | n.a. |
| 4 | 9.333 | n.a. | 2.80 | 6.0752 | 74.79 | MB* | 0.076 | n.a. | n.a. | 84379 |
| 5 | 9.983 | n.a. | 0.49 | 1.0717 | 8.55 | BM* | n.a. | n.a. | n.a. | n.a. |
| 6 | 10.119 | n.a. | 1.20 | 2.6162 | 36.68 | M* | n.a. | n.a. | n.a. | n.a. |
| 7 | 10.304 | n.a. | 86.15 | 187.0543 | 1785.20 | M* | 0.095 | 1.07 | n.a. | 65367 |
| 8 | 10.561 | n.a. | 2.79 | 6.0536 | 57.53 | M* | n.a. | n.a. | n.a. | n.a. |
| 9 | 10.658 | n.a. | 0.33 | 0.7064 | 9.77 | M* | n.a. | n.a. | n.a. | n.a. |
| 10 | 10.784 | n.a. | 0.39 | 0.8565 | 10.63 | MB* | n.a. | n.a. | n.a. | n.a. |
| 11 | 11.143 | n.a. | 0.24 | 0.5267 | 7.21 | BMB* | 0.068 | 1.32 | 3.20 | 149701 |
| 12 | 11.697 | n.a. | 0.12 | 0.2641 | 1.83 | BMB* | 0.137 | 1.55 | 5.62 | 40647 |
| 13 | 13.487 | n.a. | 4.14 | 8.9903 | 35.12 | BMB* | 0.239 | 1.37 | n.a. | 17611 |
| Sum | | 0.0000 | 100.00 | 217.1250 | 2066.08 | | | | | |

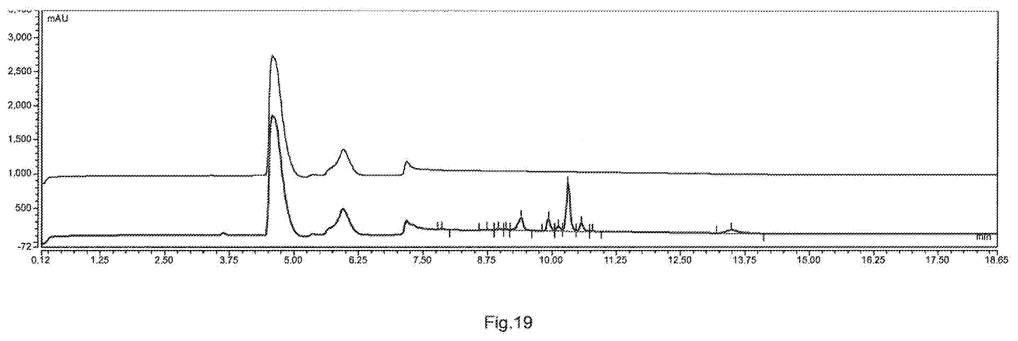

TABLE 9
Integrated peaks for FIG. 19 crude Leu-enkephaline amide (co-solvent DMSO).

| | Ret. Time | | Rel. Area | Area | Height | | Width | Asym. | Resol. | Plates |
|---|---|---|---|---|---|---|---|---|---|---|
| No. | min | Amount | % | mAU*min | mAU | Type | (50% min) | EP | EP | EP |
| 1 | 7.862 | n.a. | 0.94 | 1.2884 | 15.69 | BMB* | 0.080 | 1.14 | 3.88 | 53168 |
| 2 | 8.741 | n.a. | 1.36 | 1.8723 | 11.93 | BMB* | 0.187 | 0.94 | 0.82 | 12093 |
| 3 | 8.954 | n.a. | 2.07 | 2.8502 | 23.83 | BM* | 0.121 | n.a. | 0.96 | 30394 |
| 4 | 9.110 | n.a. | 0.80 | 1.1026 | 15.99 | MB* | 0.070 | n.a. | 1.90 | 92583 |
| 5 | 9.401 | n.a. | 16.80 | 23.0839 | 189.73 | BMB* | 0.111 | 0.82 | 3.38 | 39847 |
| 6 | 9.930 | n.a. | 10.58 | 14.5303 | 178.08 | BM* | 0.074 | 1.33 | 1.54 | 99558 |
| 7 | 10.124 | n.a. | 4.17 | 5.7331 | 71.03 | M* | 0.075 | n.a. | 1.47 | 100691 |
| 8 | 10.313 | n.a. | 44.44 | 61.0512 | 693.01 | M* | 0.077 | 1.11 | 2.01 | 98890 |
| 9 | 10.575 | n.a. | 7.77 | 10.6786 | 116.80 | M* | 0.077 | n.a. | 1.53 | 104947 |
| 10 | 10.789 | n.a. | 0.44 | 0.5992 | 6.96 | MB* | 0.088 | n.a. | 9.24 | 83131 |
| 11 | 13.483 | n.a. | 10.62 | 14.5896 | 49.88 | BMB* | 0.256 | 1.50 | n.a. | 15385 |
| Sum | | 0.0000 | 100.00 | 137.3794 | 1372.94 | | | | | |

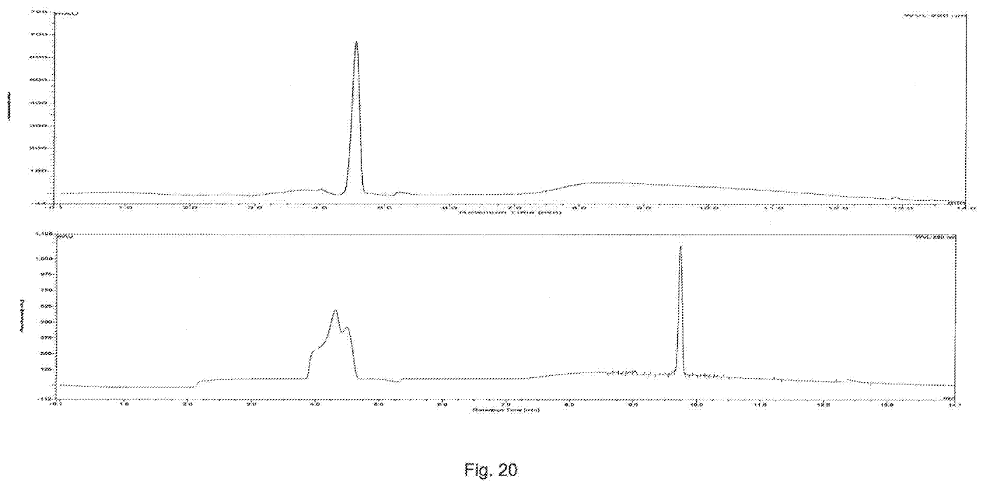

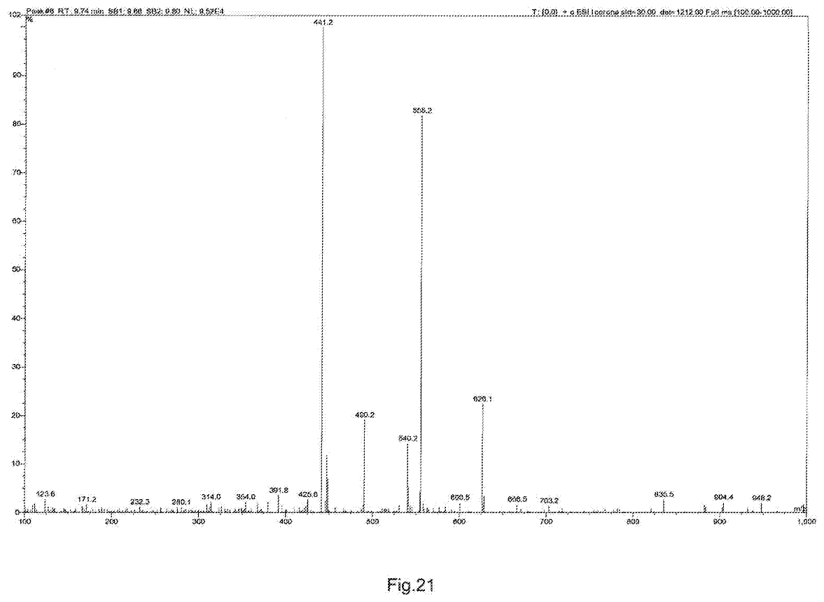

TABLE 10
Integrated peaks for FIG. 20 crude Leu-enkephalin amide (co-solvent NBP).

| | | Ret. Time | Area | | | Height | Rel. Area | |
|---|---|---|---|---|---|---|---|---|
| No. | Peakname | min | mAU*min | Amount | Type | mAU | % | Resolution |
| 1 | n.a. | 8.543 | 0.3571 | n.a. | BMB* | 7.497 | 0.46 | 2.57 |
| 2 | n.a. | 8.730 | 0.3631 | n.a. | BM* | 8.387 | 0.47 | n.a. |
| 3 | n.a. | 8.790 | 0.0398 | n.a. | MB* | 1.175 | 0.05 | n.a. |
| 4 | n.a. | 8.857 | 0.0101 | n.a. | BMB* | 0.401 | 0.01 | 1.48 |
| 5 | n.a. | 8.947 | 0.6983 | n.a. | BM* | 14.624 | 0.90 | 1.07 |
| 6 | 1 | 9.027 | 1.0477 | n.a. | MB* | 23.749 | 1.35 | 2.79 |
| 7 | n.a. | 9.213 | 0.0492 | n.a. | BMB* | 1.340 | 0.06 | 1.32 |
| 8 | n.a. | 9.300 | 0.0288 | n.a. | BMB* | 0.704 | 0.04 | 2.79 |
| 9 | n.a. | 9.510 | 0.5033 | n.a. | BMB* | 9.980 | 0.65 | n.a. |
| 10 | n.a. | 9.627 | 1.1762 | n.a. | BM* | 26.198 | 1.51 | n.a. |
| 11 | n.a. | 9.740 | 67.1712 | n.a. | M* | 1023.329 | 86.51 | n.a. |
| 12 | n.a. | 9.843 | 0.2356 | n.a. | MB* | 6.169 | 0.30 | n.a. |
| 13 | n.a. | 9.973 | 0.5089 | n.a. | BMB* | 11.893 | 0.66 | n.a. |
| 14 | n.a. | 10.107 | 0.2301 | n.a. | BM* | 5.313 | 0.30 | n.a. |
| 15 | n.a. | 10.153 | 0.1876 | n.a. | MB* | 4.222 | 0.24 | n.a. |
| 16 | n.a. | 10.230 | 0.1181 | n.a. | BMB* | 3.307 | 0.15 | 1.36 |
| 17 | n.a. | 10.343 | 0.4681 | n.a. | BMB* | 7.535 | 0.60 | 1.43 |
| 18 | n.a. | 10.477 | 0.0822 | n.a. | BMB* | 1.748 | 0.11 | 9.01 |
| 19 | n.a. | 11.290 | 0.1030 | n.a. | BMB* | 1.710 | 0.13 | 5.12 |
| 20 | n.a. | 12.387 | 4.2637 | n.a. | BMB* | 19.854 | 5.49 | n.a. |
| Total: | | | 77.6422 | 0.0000 | | 1179.133 | 100.00 | |

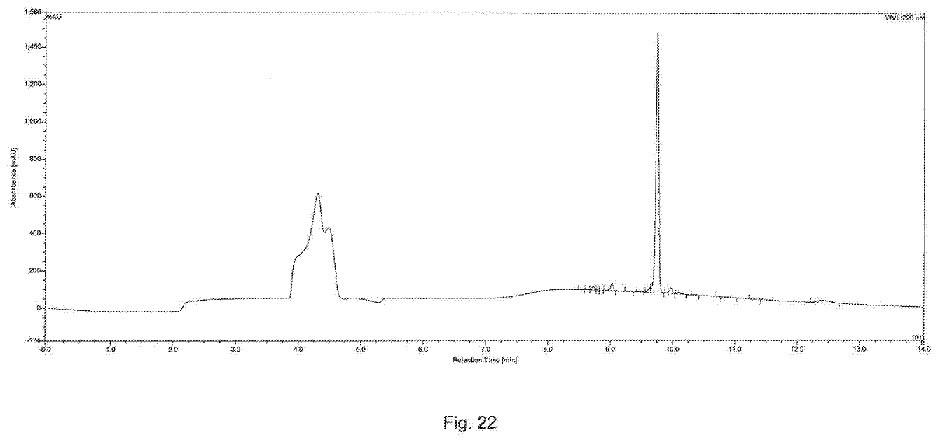

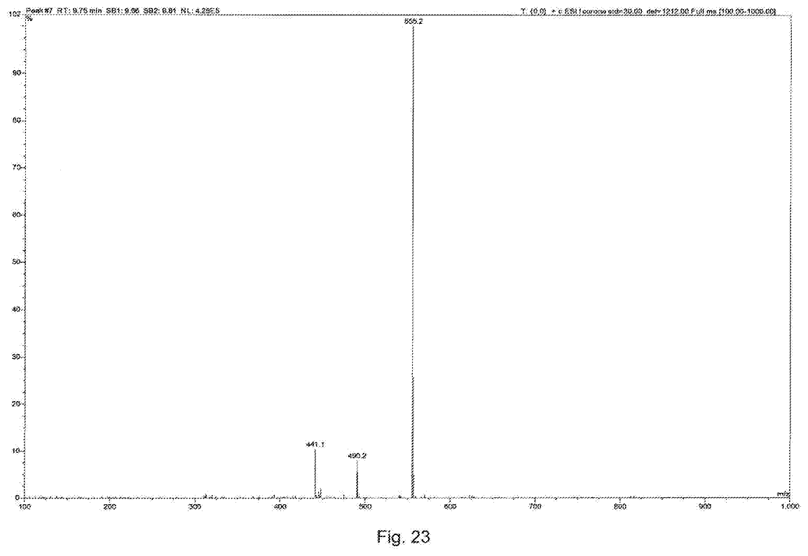

TABLE 11
Integrated peaks for FIG. 22 crude Leu-enkephalin amide (co-solvent MeCN).

| | | Ret. Time | Area | | | Height | Rel. Area | |
|---|---|---|---|---|---|---|---|---|
| No. | Peakname | min | mAU*min | Amount | Type | mAU | % | Resolution |
| 1 | n.a. | 8.547 | 0.1180 | n.a. | BMB* | 2.679 | 0.14 | 1.29 |
| 2 | n.a. | 8.643 | 0.0900 | n.a. | BM* | 2.053 | 0.11 | 1.18 |
| 3 | n.a. | 8.730 | 0.7378 | n.a. | M* | 16.544 | 0.87 | n.a. |
| 4 | n.a. | 8.787 | 0.0571 | n.a. | MB* | 1.791 | 0.07 | n.a. |
| 5 | n.a. | 8.853 | 0.0217 | n.a. | BMB* | 0.677 | 0.03 | 2.79 |
| 6 | 1 | 9.030 | 1.9630 | n.a. | BMB* | 38.991 | 2.31 | 2.59 |
| 7 | n.a. | 9.317 | 0.0825 | n.a. | BMB* | 0.981 | 0.10 | 1.43 |
| 8 | n.a. | 9.493 | 0.3760 | n.a. | BMB* | 6.614 | 0.44 | n.a. |
| 9 | n.a. | 9.630 | 1.0411 | n.a. | BM* | 23.461 | 1.22 | n.a. |
| 10 | n.a. | 9.750 | 75.2823 | n.a. | MB* | 1399.253 | 88.40 | n.a. |
| 11 | n.a. | 9.923 | 0.1379 | n.a. | BM* | 4.524 | 0.16 | n.a. |
| 12 | n.a. | 9.973 | 1.5029 | n.a. | MB* | 29.147 | 1.76 | 1.57 |
| 13 | n.a. | 10.103 | 0.6086 | n.a. | BMB* | 10.310 | 0.71 | 2.96 |
| 14 | n.a. | 10.357 | 0.2320 | n.a. | BMB* | 4.273 | 0.27 | 4.39 |
| 15 | n.a. | 10.723 | 0.0437 | n.a. | BMB* | 0.940 | 0.05 | 3.35 |
| 16 | n.a. | 10.993 | 0.0457 | n.a. | BMB* | 0.942 | 0.05 | 3.07 |
| 17 | n.a. | 11.293 | 0.1078 | n.a. | BMB* | 1.538 | 0.13 | 4.84 |
| 18 | n.a. | 12.400 | 2.7108 | n.a. | BMB* | 12.662 | 3.18 | n.a. |
| Total: | | | 85.1588 | 0.0000 | | 1557.378 | 100.00 | |

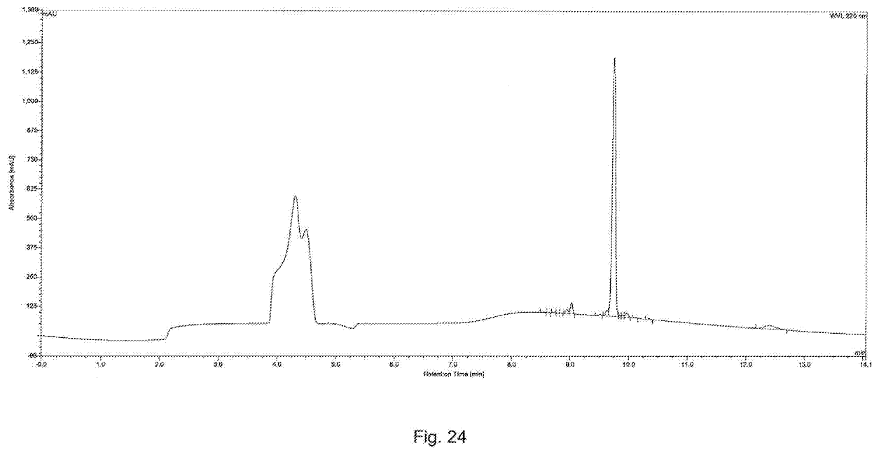

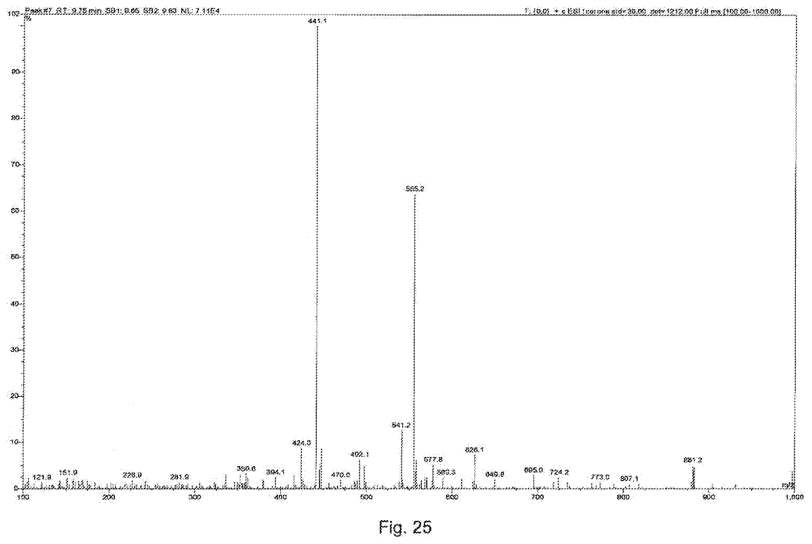

TABLE 12
Integrated peaks for FIG. 24 crude Leu-enkephalin amide (co-solvent: DMPU).

| | | Ret. Time | Area | | | Height | Rel. Area | |
|---|---|---|---|---|---|---|---|---|
| No. | Peakname | min | mAU*min | Amount | Type | mAU | % | Resolution |
| 1 | n.a. | 8.543 | 0.0985 | n.a. | BMB* | 2.215 | 0.12 | 1.41 |
| 2 | n.a. | 8.640 | 0.0147 | n.a. | BMB* | 0.395 | 0.02 | 1.36 |
| 3 | n.a. | 8.730 | 0.1814 | n.a. | BM* | 4.376 | 0.22 | n.a. |
| 4 | n.a. | 8.790 | 0.0419 | n.a. | MB* | 1.071 | 0.05 | n.a. |
| 5 | n.a. | 8.857 | 0.0275 | n.a. | BMB* | 0.899 | 0.03 | 1.34 |
| 6 | n.a. | 8.947 | 0.8268 | n.a. | BM* | 17.375 | 1.02 | 1.08 |
| 7 | 1 | 9.030 | 2.1646 | n.a. | MB* | 47.372 | 2.68 | 5.88 |
| 8 | n.a. | 9.510 | 0.3830 | n.a. | BMB* | 6.919 | 0.47 | n.a. |
| 9 | n.a. | 9.630 | 1.2136 | n.a. | BM* | 25.485 | 1.50 | n.a. |
| 10 | n.a. | 9.743 | 70.4756 | n.a. | M* | 1105.018 | 87.14 | n.a. |
| 11 | n.a. | 9.843 | 0.2877 | n.a. | M* | 6.929 | 0.36 | n.a. |
| 12 | n.a. | 9.903 | 0.3056 | n.a. | M* | 6.556 | 0.38 | n.a. |
| 13 | n.a. | 9.973 | 1.0246 | n.a. | MB* | 19.950 | 1.27 | 1.64 |
| 14 | n.a. | 10.103 | 0.1936 | n.a. | BMB* | 4.230 | 0.24 | 3.08 |
| 15 | n.a. | 10.353 | 0.2122 | n.a. | BMB* | 3.998 | 0.26 | 9.23 |
| 16 | n.a. | 12.397 | 3.4278 | n.a. | BMB* | 15.178 | 4.24 | n.a. |
| Total: | | | 80.8792 | 0.0000 | | 1267.964 | 100.00 | |

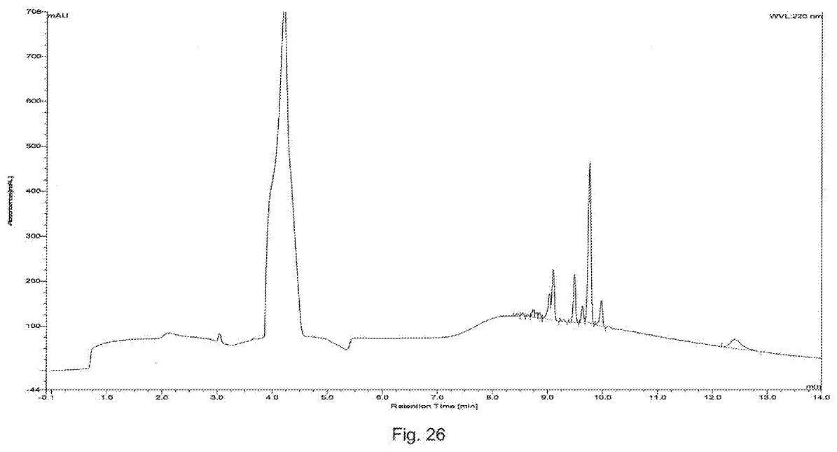

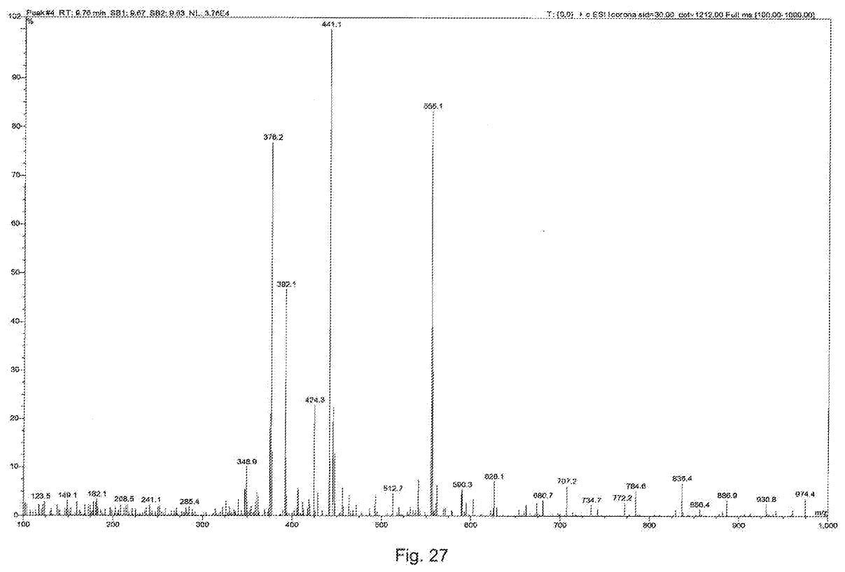

TABLE 13
Integrated peaks for FIG. 26 crude Leu-enkephalin amide (co-solvent: DMSO).

| | | Ret. Time | Area | | | Height | Rel. Area | |
|---|---|---|---|---|---|---|---|---|
| No. | Peakname | min | mAU*min | Amount | Type | mAU | % | Resolution |
| 3 | n.a. | 8.643 | 0.2050 | n.a. | BMB* | 5.220 | 0.42 | 1.22 |
| 4 | n.a. | 8.733 | 0.7436 | n.a. | BM* | 17.021 | 1.54 | n.a. |
| 5 | n.a. | 8.787 | 0.5139 | n.a. | M* | 11.606 | 1.06 | n.a. |
| 6 | n.a. | 8.853 | 0.5017 | n.a. | MB* | 11.490 | 1.04 | n.a. |
| 7 | n.a. | 9.030 | 3.2103 | n.a. | BM | 57.823 | 6.64 | n.a. |
| 8 | 1 | 9.097 | 5.6914 | n.a. | MB | 113.589 | 11.78 | 2.01 |
| 9 | n.a. | 9.280 | 0.2623 | n.a. | BMB* | 4.291 | 0.54 | 2.31 |
| 10 | n.a. | 9.487 | 5.2885 | n.a. | BMB | 107.994 | 10.95 | 1.89 |
| 11 | n.a. | 9.630 | 1.5699 | n.a. | BMb* | 35.157 | 3.25 | 1.53 |
| 12 | n.a. | 9.760 | 21.4668 | n.a. | bMB* | 357.771 | 44.43 | 2.35 |
| 13 | n.a. | 9.973 | 3.2578 | n.a. | BMB* | 57.717 | 6.74 | 10.54 |
| 14 | n.a. | 12.407 | 5.0417 | n.a. | BMB | 20.079 | 10.43 | n.a. |
| Total: | | | 48.3177 | 0.0000 | | 812.123 | 100.00 | |

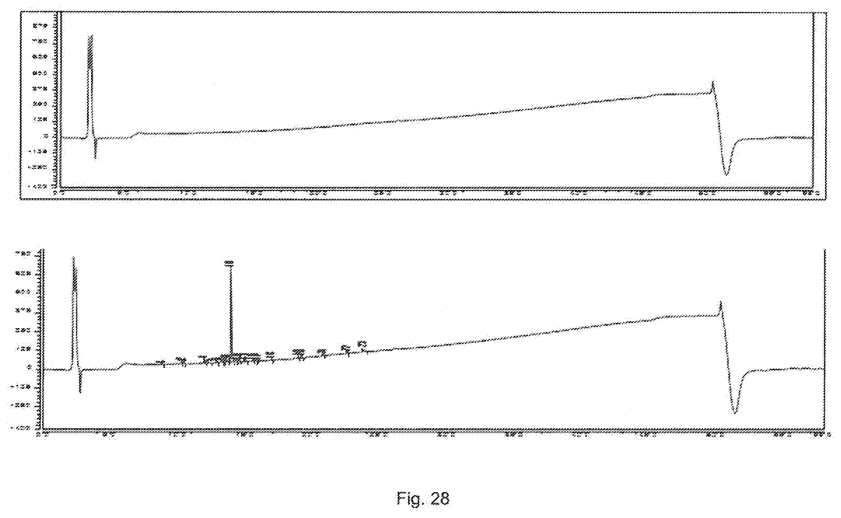

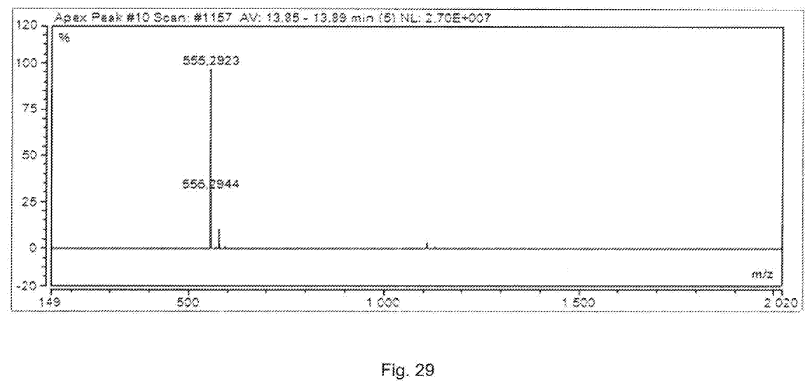

TABLE 14
Integrated peaks for FIG. 28 crude Leu-enkephalin amide

| Peak | Rt | Area | RRT | Area % |
|---|---|---|---|---|
| 1 | 8.79 | 0.190 | 0.63 | 0.34 |
| 2 | 10.26 | 0.723 | 0.74 | 1.29 |
| 3 | 10.41 | 0.236 | 0.75 | 0.42 |
| 4 | 11.93 | 1.281 | 0.86 | 2.29 |
| 5 | 12.33 | 0.174 | 0.89 | 0.31 |
| 6 | 12.91 | 0.164 | 0.93 | 0.29 |
| 7 | 13.27 | 0.203 | 0.95 | 0.36 |
| 8 | 13.39 | 0.102 | 0.96 | 0.18 |
| 9 | 13.58 | 1.260 | 0.98 | 2.25 |
| 10 | 13.87 | 45.450 | 1.00 | 81.19 |
| 11 | 14.21 | 0.507 | 1.02 | 0.91 |
| 12 | 14.41 | 0.166 | 1.03 | 0.30 |
| 13 | 14.52 | 0.608 | 1.04 | 1.09 |
| 14 | 14.93 | 0.531 | 1.07 | 0.95 |
| 15 | 15.59 | 0.174 | 1.12 | 0.31 |
| 16 | 15.69 | 0.293 | 1.13 | 0.52 |
| 17 | 15.89 | 0.114 | 1.14 | 0.20 |
| 18 | 16.92 | 0.108 | 1.22 | 0.19 |
| 19 | 18.92 | 0.232 | 1.36 | 0.41 |
| 20 | 19.14 | 0.396 | 1.38 | 0.71 |
| 21 | 20.75 | 0.164 | 1.49 | 0.29 |
| 22 | 22.53 | 0.162 | 1.62 | 0.29 |
| 23 | 23.77 | 2.741 | 1.71 | 4.90 |

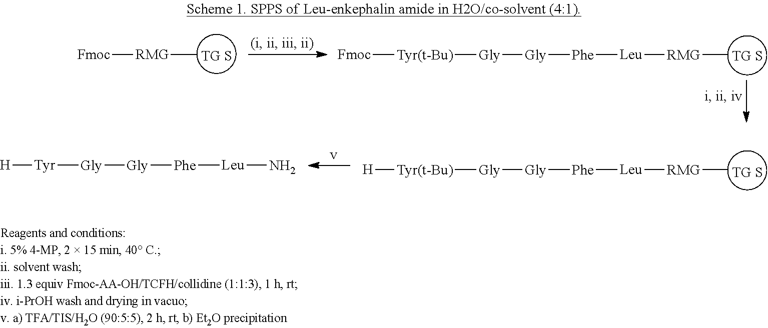

TABLE 15
Integrated peaks for FIG. 5 crude Leu-enkephalin amide.

| Peak | Rt | Area | RRT | Area % |
|---|---|---|---|---|
| 1 | 8.79 | 0.190 | 0.63 | 0.34 |
| 2 | 10.26 | 0.723 | 0.74 | 1.29 |
| 3 | 10.41 | 0.236 | 0.75 | 0.42 |
| 4 | 11.93 | 1.281 | 0.86 | 2.29 |
| 5 | 12.33 | 0.174 | 0.89 | 0.31 |
| 6 | 12.91 | 0.164 | 0.93 | 0.29 |
| 7 | 13.27 | 0.203 | 0.95 | 0.36 |
| 8 | 13.39 | 0.102 | 0.96 | 0.18 |
| 9 | 13.58 | 1.260 | 0.98 | 2.25 |
| 10 | 13.87 | 45.450 | 1.00 | 81.19 |
| 11 | 14.21 | 0.507 | 1.02 | 0.91 |
| 12 | 14.41 | 0.166 | 1.03 | 0.30 |
| 13 | 14.52 | 0.608 | 1.04 | 1.09 |
| 14 | 14.93 | 0.531 | 1.07 | 0.95 |
| 15 | 15.59 | 0.174 | 1.12 | 0.31 |
| 16 | 15.69 | 0.293 | 1.13 | 0.52 |
| 17 | 15.89 | 0.114 | 1.14 | 0.20 |
| 18 | 16.92 | 0.108 | 1.22 | 0.19 |
| 19 | 18.92 | 0.232 | 1.36 | 0.41 |
| 20 | 19.14 | 0.396 | 1.38 | 0.71 |
| 21 | 20.75 | 0.164 | 1.49 | 0.29 |
| 22 | 22.53 | 0.162 | 1.62 | 0.29 |
| 23 | 23.77 | 2.741 | 1.71 | 4.90 |

### 6. Coupling Fmoc-Gly-OH with TentaGel S NH₂ Resin in Recycled SPPS Waste

Recycling of SPPS waste from Leu-enkephalin amide synthesis described in section 5 of the experimental section:

To remove any insolubles present, the SPPS waste was filtered through a pad of celite under vacuum suction. Next, 5.0 g of an IEX resin was placed in a Buchner funnel and washed with water (3×50 mL). A 20 mL aliquot of the SPPS waste then applied onto a washed IEX resin and filtered through under vacuum suction. Four experiments were carried out:
i) SPPS waste was filtered through SP-Toyopearl-650C IEX resin (Table 16, entry 3)
ii) SPPS waste was filtered through QAE-Toyopearl-550C IEX resin (Table 16, entry 4)
iii) SPPS waste was filtered through SP-Toyopearl-650C and QAE-Toyopearl-550C IEX resins (Table 4, entry 5)
iv) SPPS waste was filtered through Amberlite MB-6113 IEX resin (Table 16, entry 6)

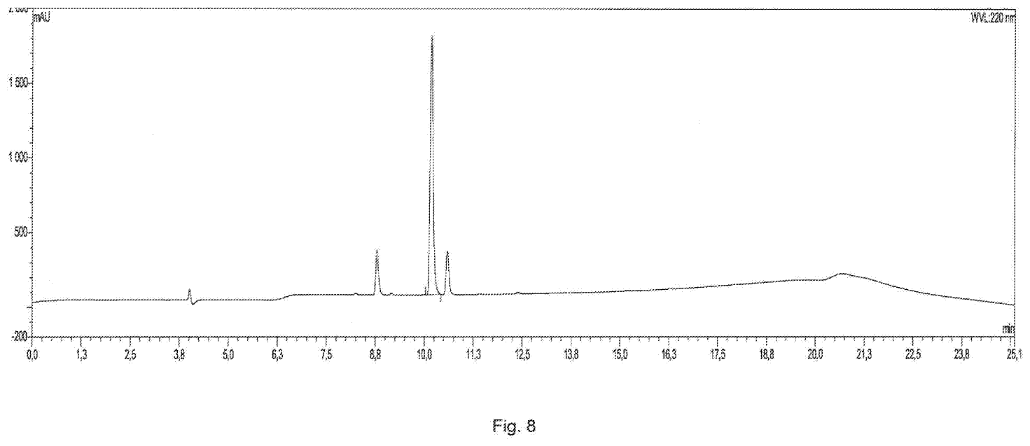

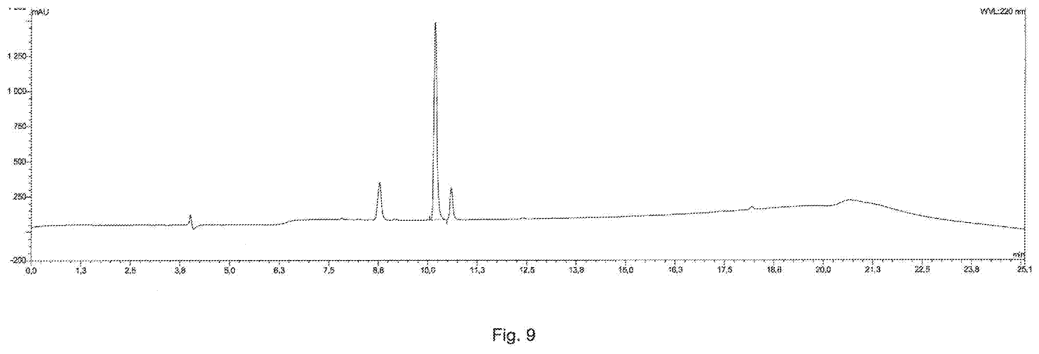

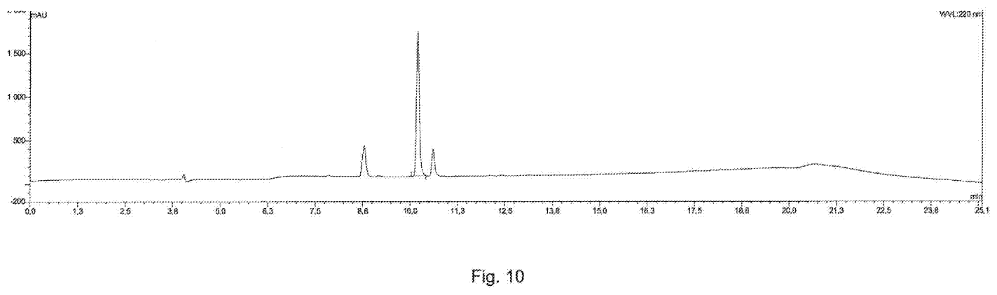

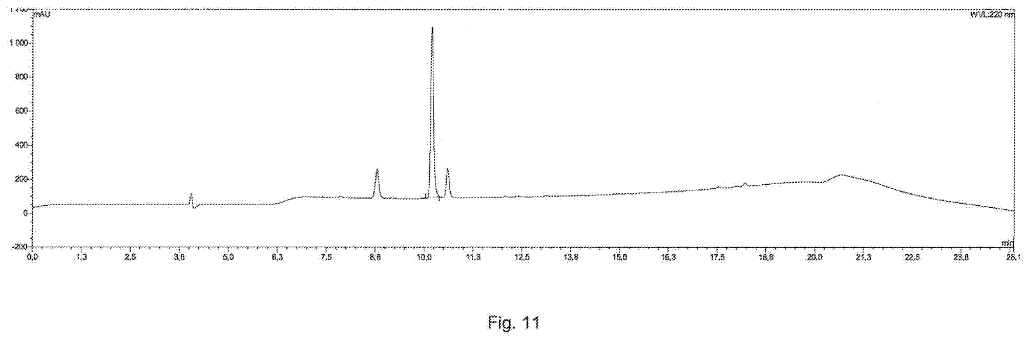

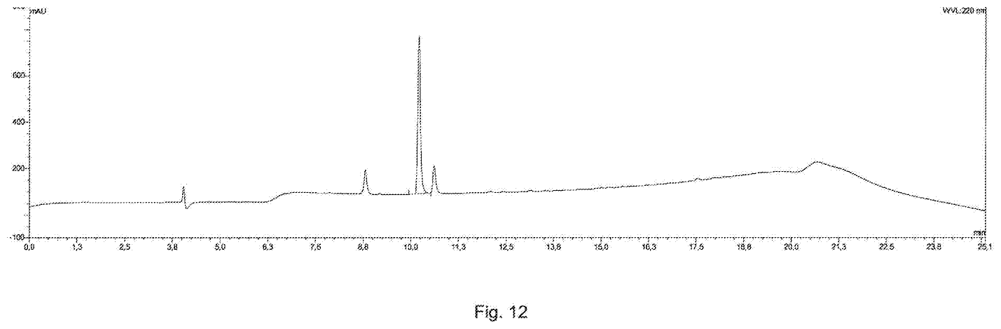

The filtrates obtained from experiments i)-iv) were analyzed by HPLC and the chromatograms thus obtained (FIGS. 10-13) were compared to the chromatograms of virgin H₂O/PC (FIG. 8) and SPPS waste (FIG. 9), respectively. The contents of PC in the samples of SPPS waste filtered through IEX resins were determined by quantifying the amounts of methyl-5-(dimethylamino)-2-methyl-5-oxopentanoate, the main constituent of PC, -using the sample of virgin PC/H₂O (FIG. 8) as a reference standard. Before using the recycled samples of SPPS waste in the test couplings of Fmoc-Gly-OH with TentaGel S NH₂ resin the content of PC was adjusted to the same content as it was in the virgin H₂O/PC (4:1).

TABLE 16
Coupling of Fmoc-Gly-OH with TG S NH₂ resin in recycled waste. ^{[a]}

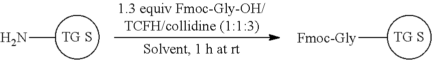

| | | Conv. |
|---|---|---|
| Entry | Solvent | (%) ^{[a]} |
| 1 | Virgin H₂O/PC (4:1) | >99^{[b]} |
| 2 | SPPS waste | 31.6 |
| 3 | SPPS waste-CEX resin-> H₂O/PC^{[c]} | 80.6 |
| 4 | SPPS waste-AEX resin-> H₂O/PC^{[c]} | 85.5 |
| 5 | SPPS waste-CEX and AEX resins-> | >99^{[b]} |
| | H₂O/PC^{[c]} | |
| 6 | SPPS waste-mixed IEX resin-> | >99^{[b]} |
| | H₂O/PC^{[c]} | |

^{[a]} Determined by quantifying Fmoc content on Fmoc-Gly TG S resins;
^{[b]}Coupling complete according to a negative (ninhydrin) color test;
^{[c]}H₂O/PC ratio was adjusted to 4:1.

### 7. SPPS of Leu-Enkephalin Amide in Recycled SPPS Waste

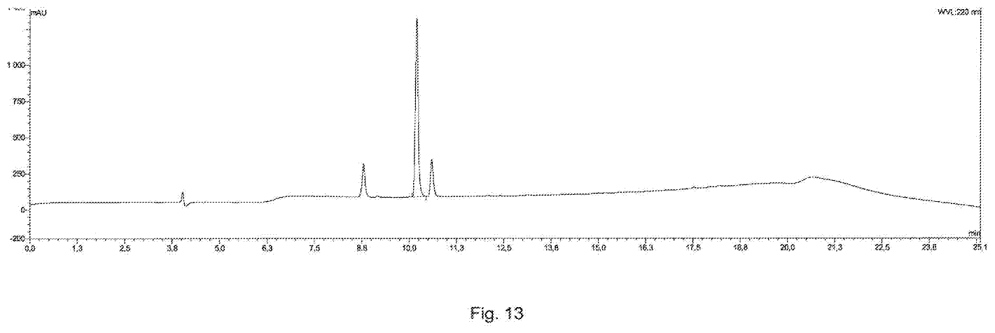

The synthesis was carried out in the same manner as described in section 5. The scale used was 50% of the original scale i.e. 0.5 g of 0.18M Fmoc-RMG TentaGel S starting resin (0.09 mmol) was used. All amounts of the reagents, reactants and solvents were scaled down accordingly whereas the reaction times and temperatures were kept the same. The requisite PC/H₂O (1:4) was obtained according to the procedure described in section 6. Upon completion of the synthesis the final resin was washed with i-PrOH and dried to constant weight in vacuo affording 0.53 g of Leu-enkephalin amide RMG TentaGel S peptide resin. The total amount of H₂O/PC containing SPPS waste was about 185 mL. 100 mg of the Leu-enkephalin amide resin was weighed into a fritted syringe, 1.0 mL of TFA/TIS/H₂O (90:5:5) was added and the resulting slurry was shaken at rt for 2 h. The spent resin was filtered off and washed with 2×0.5 mL TFA. The combined volatiles were removed in vacuo and the crude peptide was precipitated by 2×20 mL diethyl ether (Et₂O) affording 9.6 mg (84%) of crude Leu-enkephalin amide. The HPLC purity of the crude product was 86% (FIG. 13 and Table 17).

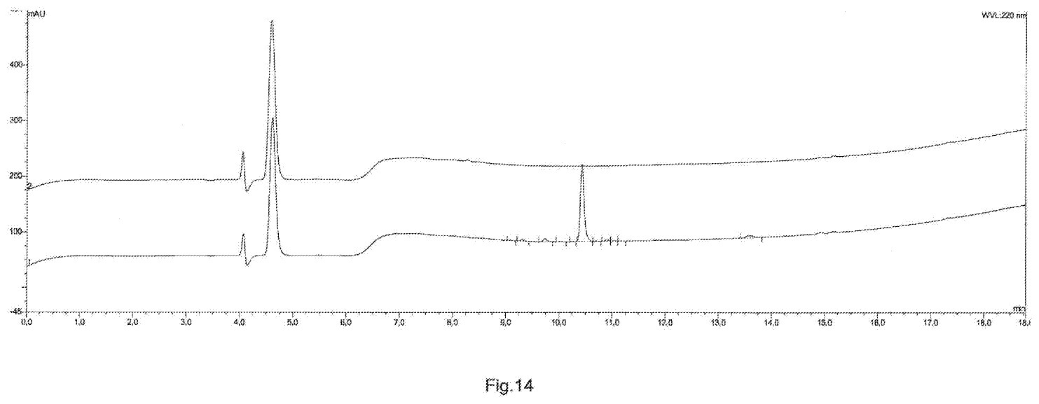

TABLE 17
Integrated peaks for FIG. 14 crude Leu-enkephalin amide.

| | | Ret. Time | Area | | | Height | Rel. Area | |
|---|---|---|---|---|---|---|---|---|
| No. | Peakname | min | mAU*min | Amount | Type | mAU | % | Resolution |
| 1 | n.a. | 9.095 | 0.0236 | n.a. | BMB* | 0.318 | 0.18 | 1.77 |
| 2 | n.a. | 9.305 | 0.2220 | n.a. | BMB* | 2.999 | 1.69 | 3.54 |
| 3 | n.a. | 9.735 | 0.4301 | n.a. | BMB* | 5.340 | 3.28 | 2.37 |
| 4 | n.a. | 10.024 | 0.0382 | n.a. | BMB* | 0.515 | 0.29 | 2.15 |
| 5 | n.a. | 10.262 | 0.0375 | n.a. | BMB* | 0.616 | 0.29 | 1.49 |
| 6 | n.a. | 10.430 | 11.3343 | n.a. | BMB* | 138.777 | 86.36 | 2.30 |
| 7 | n.a. | 10.696 | 0.0511 | n.a. | BMB* | 0.792 | 0.39 | 1.74 |
| 8 | n.a. | 10.897 | 0.1951 | n.a. | BM* | 2.535 | 1.49 | 1.12 |
| 9 | n.a. | 11.041 | 0.0972 | n.a. | M* | 1.158 | 0.74 | n.a. |
| 10 | n.a. | 11.149 | 0.0448 | n.a. | MB* | 0.541 | 0.34 | n.a. |
| 11 | n.a. | 13.585 | 0.6510 | n.a. | BMB* | 4.169 | 4.96 | n.a. |
| Total: | | | 13.1249 | 0.0000 | | 157.759 | 100.00 | |

1-43. (canceled)

44. A solid-phase peptide synthesis method comprising the provision of an activated Fmoc-α-amine protected amino acid moiety and an Fmoc-α-amine protected peptide fragment bound to a resin;
deprotecting the Fmoc-α-amine protected peptide fragment bound to the resin;
coupling of the activated Fmoc-α-amine protected amino acid moiety with the deprotected Fmoc-α-amine protected peptide fragment bound to the resin thereby forming a peptide bond;
wherein the amide (peptide) coupling is performed in an aqueous solution comprising at least one organic co-solvent miscible with water, the aqueous solution capable of sufficiently solubilize the activated Fmoc-α-amine protected amino acid moiety, and wherein the resin is capable of swelling in the presence of the aqueous solution above about 4 mLg⁻¹ (based on weight resin) thereby elongating the amino acid fragment bound to the resin.

45. The method according to claim 44, wherein co-solvent is a polar aprotic co-solvent.

46. The method according to claim 44, wherein the activated Fmoc-α-amine protected amino acid moiety is activated in a separate step preceding the coupling, or that the activation is formed in situ; and that the amide-coupling is performed in the presence of at least a coupling agent (CA).

47. The method according to claim 46, wherein the amide-coupling is performed in the presence of a base, suitably selected form alkyl derivatives of pyridine such as alkyl derivatives of pyridine selected from picoline, lutidine and collidine and any regioisomers thereof, specifically methyl derivatives of pyridine

48. The method according to claim 45, wherein the polar aprotic co-solvent is selected from co-solvents of the chemical structure (1) 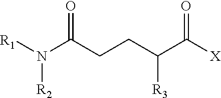
where R₁, R₂, and R₃ are independently selected from alkyls having from 1 to 3 carbon atoms, and if X is an oxygen atom, then one alkyl group comprising from 1 to 3 carbon atoms is bound to the oxygen atom; and if X is a nitrogen atom, then two alkyl groups are bound to the nitrogen atom, the alkyl group(s) independently selected from 1 to 3 carbon atoms.

49. The method according to claim 48, wherein R₁, R₂, and R₃ are all methyl groups, and the alkyl group(s) bound to the oxygen or nitrogen is(are) methyl groups.

50. The method according to claim 44, wherein the resin comprises polyethylene glycol.

51. The method according to claim 44, wherein the resin comprises polystyrene and polyethylene glycol.

52. The method according to claim 44, wherein the resin is a polystyrene-polyethylene glycol graft co-polymer.

53. The method according to claim 44, wherein the resin is a polystyrene-polyethylene graft co-polymer comprising cross-linked polystyrene and polyethylene glycol bound via an ether-link to the cross-linked polystyrene.

54. The method according to claim 50, wherein the polyethylene glycol has a MW in the range of from 1000 Da to 5000 Da.

55. The method according to claim 50, wherein the ratio of polyethylene glycol to total weight of resin is above about 50 wt %.

56. The method according to claim 46, wherein the CA is selected from the group consisting of carbodiimides, N-hydroxylamine-based CAs, uronium (amidium) based CAs, phosphonium based CAs, compounds converting acids to acid chlorides, compounds converting carboxylic acids to the corresponding acyl fluorides, and triazine-based CAs.

57. The method according to claim 46, wherein the CA is selected from the group consisting of

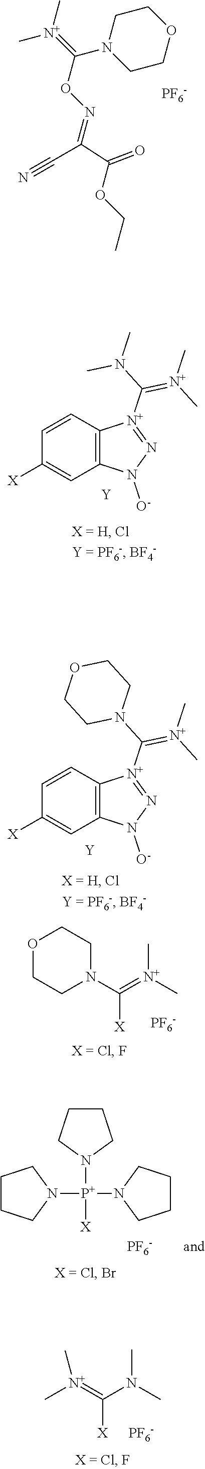

58. The method according to claim 46, wherein the CA is selected from

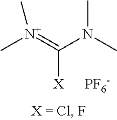

59. The method according to claim 47, wherein the base is selected from the group consisting of aliphatic amines, aromatic amines, trimethyl derivatives of pyridine, imidazole, N-methylimidazole (NMI) and inorganic bases.

60. The method according to claim 47, wherein the base is selected from the group consisting of diisopropylethylamine (DIEA), N-methylmorpholine (NMM), trimethyl derivatives of pyridine, pyridine, lutidine, and inorganic bases including phosphates, carbonates, sulfates, acetates, borates in their lithium, sodium, potassium, calcium or tetraalkylammonium forms.

61. The method according to claim 47, wherein the base is trimethyl derivatives of pyridine such as picoline, lutidine and collidine.

62. A solid-phase peptide synthesis method comprising the provision of an activated Fmoc-α-amine protected amino acid moiety and an Fmoc-α-amine protected peptide fragment bound to a resin;
deprotecting the Fmoc-α-amine protected peptide fragment bound to the resin;
coupling of the activated Fmoc-α-amine protected amino acid moiety with the deprotected Fmoc-α-amine protected peptide fragment bound to the resin thereby forming a peptide bond;
wherein the amide (peptide) coupling is performed in an aqueous solution comprising at least one organic co-solvent miscible with water, the aqueous solution capable of sufficiently solubilize the Fmoc-α-amine protected amino acid or Fmoc-α-amine protected peptide fragment, and wherein the resin is capable of swelling in the presence of the aqueous solution above about 4 mLg⁻¹ (based on weight resin) thereby forming an elongated peptide fragment bound to the resin; wherein the activation of the protected amino acid is performed in the presence of a coupling agent and a base, the organic co-solvent having the structural formula: 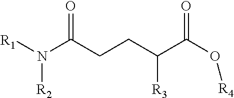
where R₁, R₂, R₃ and R₄ independently selected from alkyls having from 1 to 3 carbon;
the coupling agent being selected from the 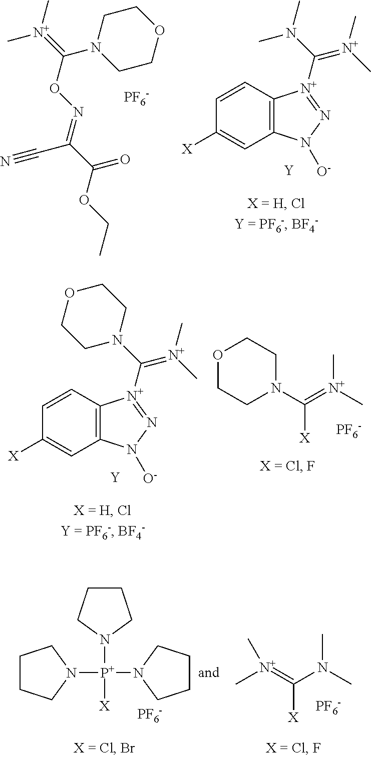
the base being selected from trimethyl derivatives of pyridine;
wherein the resin is selected from copolymers of styrene and ethylene glycol.
